(19) United States
(12) Patent Application Publication
(10) Pub. No.: US 2026/0363732 A1
(43) Pub. Date: Nov. 19, 2026

(54) MOTORIZED FLUID-DELIVERY SKIN-TREATMENT SYSTEM

(71) Applicants: Franklin J. Chang, Pasadena, CA (US); Henry Ping Chang, Pasadena, CA (US)

(72) Inventors: Franklin J. Chang, Pasadena, CA (US); Henry Ping Chang, Pasadena, CA (US)

(21) Appl. No.: 19/749,497

(22) Filed: Jul. 21, 2026

Related U.S. Application Data
(63) Continuation-in-part of application No. 19/071,679, filed on Mar. 5, 2025, now Pat. No. 12,733,956.

(51) Int. Cl. A61B 17/54; A61B 17/00; A61M 1/00

(52) U.S. Cl. CPC A61B 17/545; A61M 1/774; A61B 2017/00761

(57) ABSTRACT

A skin-treatment system includes a treatment-fluid reservoir, variable-rate treatment-fluid pump, vacuum source, waste receptacle, motorized handpiece, rotatable treatment tip, motor-current sensor, rotational-speed sensor, and electronic controller. The treatment tip includes distal contact portions, a fluid outlet, a vacuum inlet, and a recessed passage whose floor remains separated from skin while the distal contact portions touch the skin. Before treatment, the controller primes the treatment-fluid path and calculates baseline motor current and rotational speed. During treatment, the controller compares an increase in motor current and a decrease in rotational speed with stored thresholds. When both thresholds are exceeded for successive comparisons, the controller reduces rotational speed and increases treatment-fluid delivery. If motor current does not decrease by a stored amount, the controller stops rotation, reduces suction, and continues treatment-fluid delivery for a release interval. Interchangeable treatment faces include ridges and channels, projecting members, a planar contact land, and bonded abrasive particles.

### CROSS-REFERENCE TO RELATED APPLICATIONS

This application is a continuation-in-part of U.S. patent application Ser. No. 19/071,679, filed Mar. 5, 2025, the disclosure of which is incorporated herein by reference. The disclosures of U.S. Pat. Nos. 9,186,490 and 9,044,582 are also incorporated herein by reference.

### FIELD OF THE INVENTION

The present disclosure relates to devices and methods for microdermabrasion and hydrodermabrasion. More particularly, the disclosure relates to motorized treatment tips, treatment-fluid-delivery and vacuum-return structures, interchangeable skin-contact surfaces, and control of motor speed, treatment-fluid delivery, and suction in response to measured motor current and measured rotational speed.

### BACKGROUND OF THE INVENTION

U.S. Pat. No. 9,186,490 describes a skin-treatment device including a handpiece and a removable tip having a skin-contact surface. A fluid-delivery structure at the skin-contact surface includes a fluid aperture and a vacuum inlet. A fluid path between the fluid aperture and the vacuum inlet increases the distance traveled by fluid across the skin-contact surface. An abrading structure, an electrode structure, and a microneedling structure can be provided with the fluid-delivery structure.

U.S. Pat. No. 9,044,582 describes an apparatus for transdermal fluid delivery including a handpiece and a movable tip having a skin-contact surface. A fluid-delivery structure includes an aperture at the skin-contact surface and a vacuum inlet. An abrading structure, an electrode structure, and a microneedling structure can be provided with the fluid-delivery structure. Treatment fluid passes across the skin-contact surface, interacts with the abrading structure and the electrodes, and is withdrawn through a return path.

A need remains for treatment-fluid delivery that maintains fluid at the skin-contact region and moves used treatment fluid and removed material to a vacuum-return path.

A skin surface may carry native sebum and other hydrophobic lipids that resist uniform wetting by an aqueous treatment fluid. Under some conditions, compliant skin can seal against a treatment face, restrict movement of treatment fluid or air, and increase friction between the treatment face and the skin.

Static treatment tips and manually translated treatment tips may require repeated passes or changes in direction to treat a region. A motorized tip can provide localized movement, but fixed motor, vacuum, and pump settings may not accommodate changes in skin contour, skin hydration, skin compliance, treatment-fluid quantity, or accumulation of skin material and treatment fluid on the treatment face.

Accordingly, a need exists for a treatment system that maintains treatment-fluid movement across a rotating skin-contact face, decreases rotational speed and increases treatment-fluid delivery when measured motor current rises and measured rotational speed falls, and supports both continuous linear treatment and stationary treatment with interchangeable treatment surfaces.

### SUMMARY OF THE INVENTION

One embodiment includes an abrasive motor-driven treatment tip having molded grooves and surface structures and coupled to a spindle shaft. The treatment tip can be selected to provide a selected exfoliation level during a treatment pass. One or more openings in the treatment tip provide a treatment-fluid outlet, a vacuum inlet, or both.

In one aspect, a treatment tip for a microdermabrasion device includes one or more treatment-fluid openings and one or more molded grooves. The treatment tip is formed from a polymer and carries an abrasive coating.

The abrasive tip has an adhesive, such as epoxy resin, and an abrasive such as emery sand.

In one aspect, a skin-treatment system includes a console having a treatment-fluid reservoir, a variable-rate treatment-fluid pump, a vacuum source, a waste receptacle, and a processor. A motorized handpiece is coupled to the console by a treatment-fluid supply path, a vacuum-return path, and electrical conductors. A removable treatment tip is driven by an electric motor in the handpiece and includes a skin-contact face, a fluid outlet, and a vacuum inlet.

In some embodiments, treatment fluid is delivered to the skin-contact face while the face rotates and suction is applied. The treatment fluid moves through a recessed channel, an annular recess, or a clearance between the face and the skin and is withdrawn with removed material through a vacuum-return path. Rotation moves the treatment fluid along the skin and can disperse native surface lipids into the treatment fluid while treatment fluid remains in recessed portions of the face.

In some embodiments, a motor-current sensor measures current supplied to the handpiece motor, and a rotational-speed sensor measures rotation of the drive member or treatment tip. The processor calculates a filtered motor-current value from successive motor-current samples, subtracts a baseline motor-current value from the filtered motor-current value to obtain a motor-current increase, and subtracts a measured rotational speed from a commanded rotational speed to obtain a rotational-speed decrease. When the motor-current increase exceeds a stored positive motor-current-increase threshold and the rotational-speed decrease exceeds a stored positive rotational-speed-decrease threshold for more than one successive comparison, the processor sends a lower rotational-speed command to the motor driver and a higher treatment-fluid-delivery command to the pump driver.

The skin-contact face can include raised ridges separated by recessed channels, projecting pins or nodules, a planar contact land adjacent to a recess, an abrasive-particle coating, or a combination of these structures. In a grooved embodiment, an inclined sidewall of each raised ridge can form an angle from 35 degrees to 60 degrees relative to a plane containing distal ends of the raised ridges, each recessed channel can have a depth measured from that plane to a floor of the recessed channel from 0.2 millimeter to 0.7 millimeter, and adjacent raised ridges can have a center-to-center spacing from 0.5 millimeter to 1.2 millimeters.

In some embodiments, a treatment tip has a coaxial arrangement including an inner structure that provides treatment-fluid delivery and vacuum return, an intermediate structure that carries the skin-contact face, and an outer structure that carries one or more electrodes. The structures can be removable together or separately and can be disposable, reusable, or a combination of disposable and reusable structures.

In some embodiments, a removable treatment-fluid container is supported by the handpiece. A noncircular first treatment-fluid conduit and a bypass conduit route treatment fluid beside a motor compartment to a core inlet while fluid-tight walls isolate the motor compartment from the treatment fluid and the vacuum-return stream.

The treatment tip can be moved in a continuous linear stroke or held at one treatment location. Rotation of the treatment face moves surface structures in circumferential directions across the skin-contact region, and suction withdraws treatment fluid, surface material, and material released from follicular openings.

In one embodiment, the processor and a memory form an electronic controller. The memory stores a first motor-current threshold, a reset motor-current threshold lower than the first motor-current threshold, a selected motor-speed command, and a selected pump-delivery command. When sampled motor current exceeds the first motor-current threshold, the controller sends a lower motor-speed command to a motor driver and a higher pump-delivery command to a pump driver. The controller maintains the changed commands until sampled motor current falls below the reset motor-current threshold.

In one embodiment, a fluid outlet and a vacuum inlet are separated across the skin-contact face by a recessed channel or an annular recess. The variable-rate pump supplies treatment fluid to the fluid outlet, and the vacuum source draws the treatment fluid through the recessed channel or annular recess to the vacuum inlet. The resulting fluid volume occupies the recess between the treatment tip and the skin while the treatment tip rotates.

In one embodiment, a motor-current sensor measures current supplied to the handpiece motor at a sampling rate greater than 100 hertz. A second motor-current threshold greater than the first motor-current threshold causes the controller to stop the motor when increased treatment-fluid delivery and reduced motor speed do not lower the measured motor current.

In one embodiment, the processor establishes a baseline motor-current value and a baseline rotational-speed value while the treatment tip is spaced from the skin and while the motor, treatment-fluid pump, and vacuum source operate at selected settings. The processor calculates the baseline values from successive sensor samples obtained after treatment fluid has reached the vacuum-return path.

During skin contact, the processor subtracts the baseline motor-current value from a filtered motor-current value and subtracts a measured rotational speed from a commanded rotational speed. The processor changes the motor and pump commands only when the motor-current increase exceeds a positive stored motor-current-increase threshold and the rotational-speed decrease exceeds a positive stored rotational-speed-decrease threshold for a stored number of successive comparisons greater than one.

After both stored thresholds have been exceeded for the stored number of successive comparisons, the processor stores the filtered motor-current value from the last comparison as a triggering motor-current value. Before acquiring a subsequent motor-current sample, the processor sends a second rotational-speed command specifying a lower speed and a second pump-delivery command specifying a higher treatment-fluid delivery rate, begins a stored response interval, and maintains the vacuum source at the selected suction setting.

At the end of the response interval, the processor calculates a post-response filtered motor-current value and subtracts that value from the triggering motor-current value. When the resulting reduction is smaller than a stored positive minimum motor-current reduction, or when the post-response filtered motor-current value reaches a stored motor-stop threshold, the processor stops the motor, sends a suction command that produces less suction than the selected suction setting, and continues treatment-fluid delivery for a stored nonzero release interval.

In one handpiece embodiment, fluid-tight walls separate a motor compartment from a noncircular treatment-fluid conduit, a bypass conduit connected to the noncircular treatment-fluid conduit, and a vacuum-return passage. The noncircular treatment-fluid conduit has a concave wall facing the motor compartment and a convex wall facing the exterior of the handpiece so that the conduit occupies space beside the motor without entering the motor compartment.

### BRIEF DESCRIPTION OF THE DRAWINGS

FIG. 1 illustrates a device.

FIG. 2 is a cross-sectional view of the device.

FIG. 3 is an exploded view of the device.

FIGS. 4 through 6 illustrate an embodiment having a serum attachment.

FIG. 7 is a detailed view of the core of the device.

FIG. 8 is an exploded view of the core, shaft, motor, and tube couplings.

FIG. 9 is a top view of the core and shaft.

FIG. 10 schematically illustrates fluid entering through inlet 42, entering shaft 50 through aperture 56, leaving through opening 52, contacting the skin, passing through gap 25, entering suction port 48, leaving through outlet port 49, and passing to tube 90.

FIG. 11 illustrates an embodiment in which the device includes electrodes 112 and 114 on the tip cover.

FIG. 12 is an exploded view of the tip cover, tip, and core of the embodiment of FIG. 11.

FIG. 13A illustrates operation of the tip.

FIG. 13B is a cross-sectional view illustrating operation of the tip.

FIG. 14A illustrates operation of the abrasive tip.

FIG. 14B is a cross-sectional view illustrating operation of the abrasive tip.

FIG. 15A is an enlarged schematic view of a treatment tip having raised ridges and recessed fluid channels. The view identifies an angle formed by an inclined sidewall of a raised ridge relative to a plane containing distal ridge ends, a recessed-channel depth measured from that plane to a channel floor, a center-to-center spacing between adjacent ridges, a first channel direction, and a second channel direction different from the first channel direction. FIGS. 15A through 15D are arranged on one drawing sheet and use the same treatment-tip reference numerals.

FIG. 15B is an enlarged schematic view of a treatment tip having projecting pins or projecting nodules, each having a base joined to the treatment face and a distal skin-contact end, with fluid passages between adjacent projecting pins or projecting nodules.

FIG. 15C is an enlarged schematic view of a treatment tip having a continuous planar skin-contact land, an annular recessed passage adjacent to the planar skin-contact land, a fluid outlet, and a vacuum inlet.

FIG. 15D is an enlarged schematic view of a treatment tip having a substrate, an adhesive layer, abrasive particles bonded to the adhesive layer, and at least one molded fluid channel connecting an outlet region with an inlet region.

FIG. 16A is a longitudinal sectional view of the handpiece showing the handpiece housing 217, proximal connector 224, distal opening 226, treatment-fluid container 231, treatment-fluid-container interface 230, puncturing member 290, annular container seal 292, first treatment-fluid conduit 232, bypass conduit 234, fluid-tight wall 229, motor compartment 228, electric motor 218, motor driver 268, rotatable drive member 220, core inlet 233, vacuum-return passage 235, motor-current sensor 270, rotational-speed sensor 278, electrical connector 288, treatment-fluid supply seal 294, rotary seal 296, and an acute angle between a longitudinal container axis and a longitudinal handpiece axis.

FIG. 16B is a transverse sectional view through the motor compartment showing the concave inner wall and convex outer wall of the noncircular first treatment-fluid conduit 232, the bypass conduit 234, the vacuum-return passage 235, and the fluid-tight wall 229 that separates those passages from the motor compartment 228.

FIG. 16C is an enlarged cross-sectional view of a rotating treatment tip and skin showing distal contact portions of the treatment tip touching the skin, a fluid outlet, a recessed passage containing treatment fluid, a vacuum inlet, a follicular opening, material within the follicular opening, and directions of treatment-tip rotation, treatment-fluid movement, and vacuum-return movement.

FIG. 17 is a functional block diagram of the treatment-fluid reservoir, variable-rate treatment-fluid pump, pump driver, vacuum source, vacuum-control actuator, waste receptacle, electric motor, motor driver, motor-current sensor, signal-conditioning circuit, analog-to-digital converter, rotational-speed sensor, processor, memory, user control, status indicator, and electrical and fluid connections between those components. The diagram also shows baseline measurement before skin contact, comparison of a motor-current increase and a rotational-speed decrease with stored numerical thresholds, storage of a triggering motor-current value, issuance of a lower rotational-speed command and a higher treatment-fluid-delivery command before acquisition of a subsequent motor-current sample, calculation of a post-response motor-current reduction, incremental return toward selected commands, and a motor-stop, suction-reduction, and treatment-fluid-release sequence.

### DETAILED DESCRIPTION OF THE INVENTION

The device can combine abrasive peeling, electrical stimulation, and treatment-fluid delivery during a treatment procedure. The selected structures and operating settings determine whether the procedure affects the stratum corneum, increases movement of a treatment fluid through superficial pathways, applies electrical stimulation, or performs a combination of those operations.

A microdermabrasion device can include the following physical components and operating features:

A motor rotates a removable treatment tip at a selected speed from 10 to 300 revolutions per minute for an operating mode directed to exfoliation and treatment-fluid delivery.

An abrasive coating is applied to a treatment surface by electrostatic deposition and is cured on the treatment surface.

A treatment-fluid supply path and a vacuum-return path extend through a rotating treatment tip, and a stationary tip cover defines a vacuum space around the rotating treatment tip.

Interchangeable treatment tips provide an abrasive surface, one or more electrodes, a treatment-fluid passage, a vacuum-return passage, or a combination of those structures.

The handpiece has an ergonomic housing and a removable treatment-fluid-container interface.

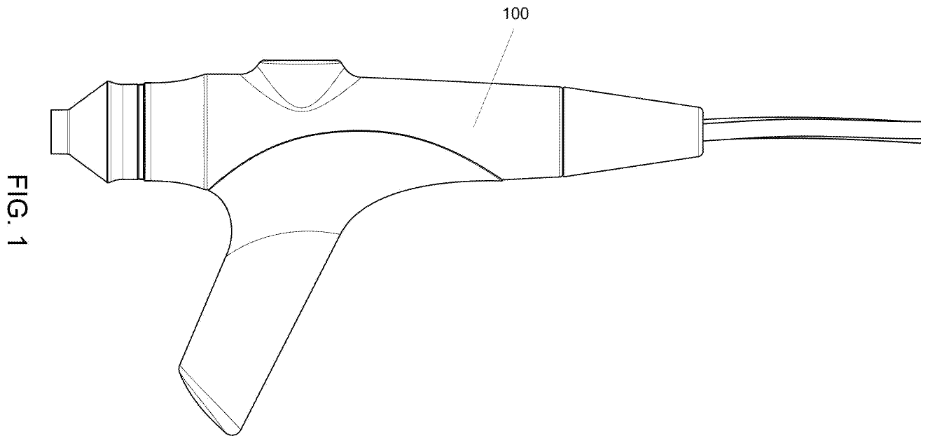

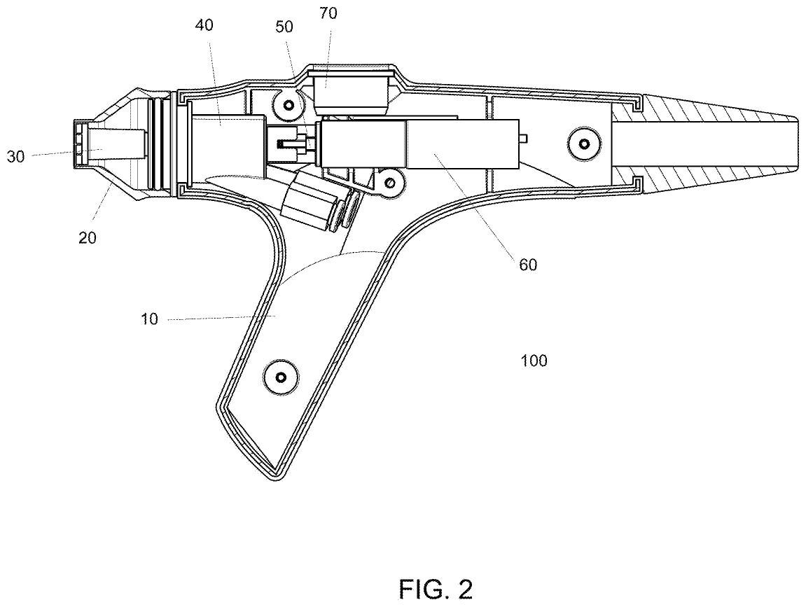

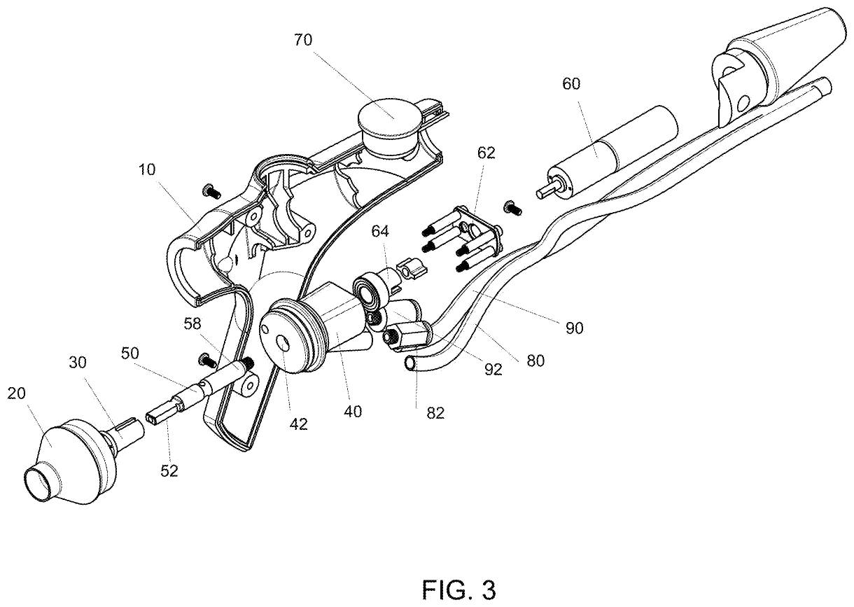

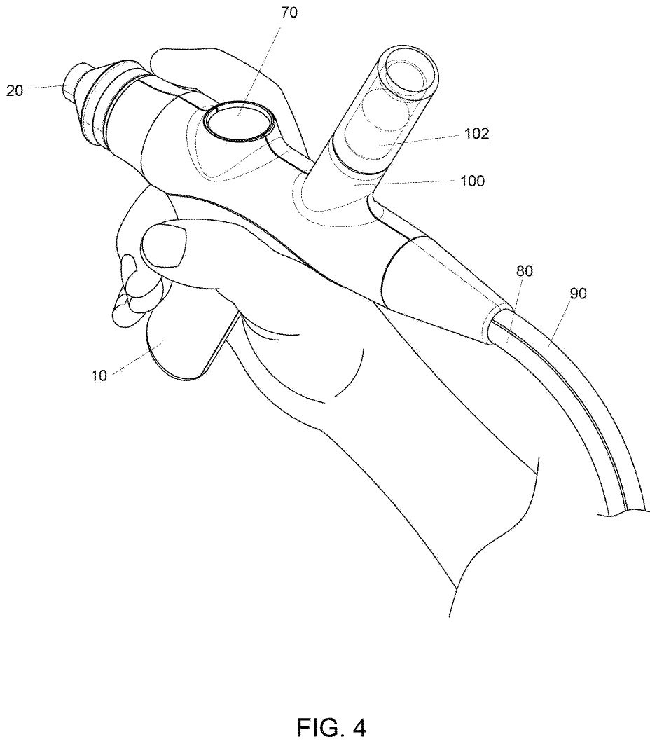

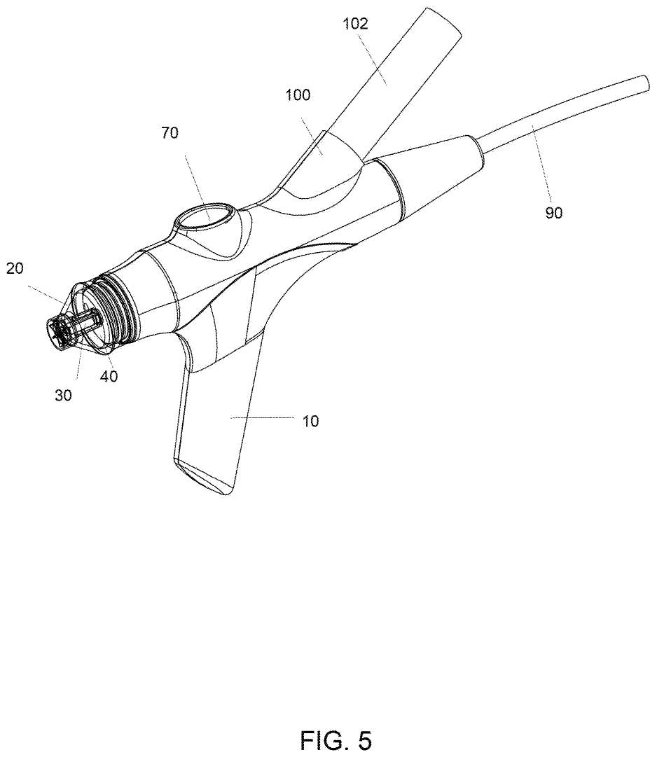

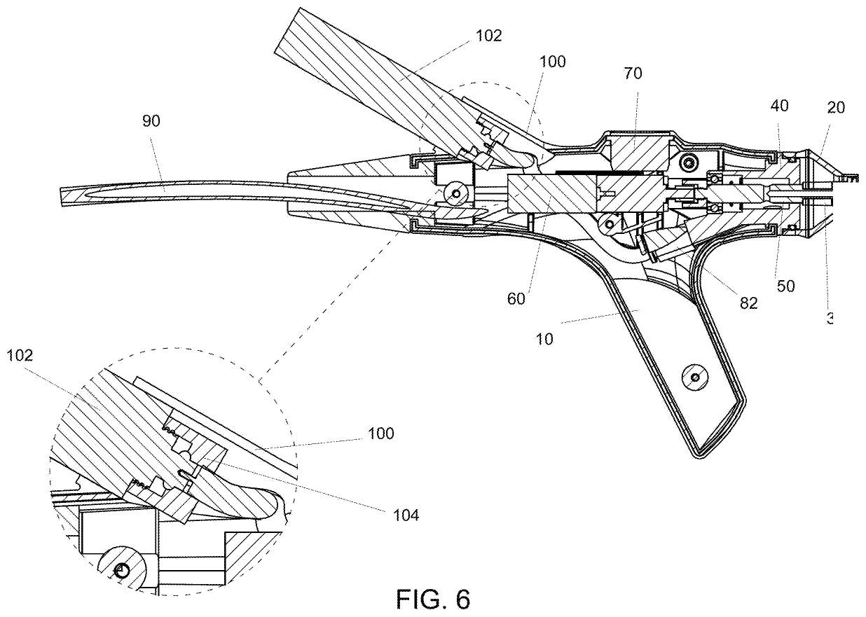

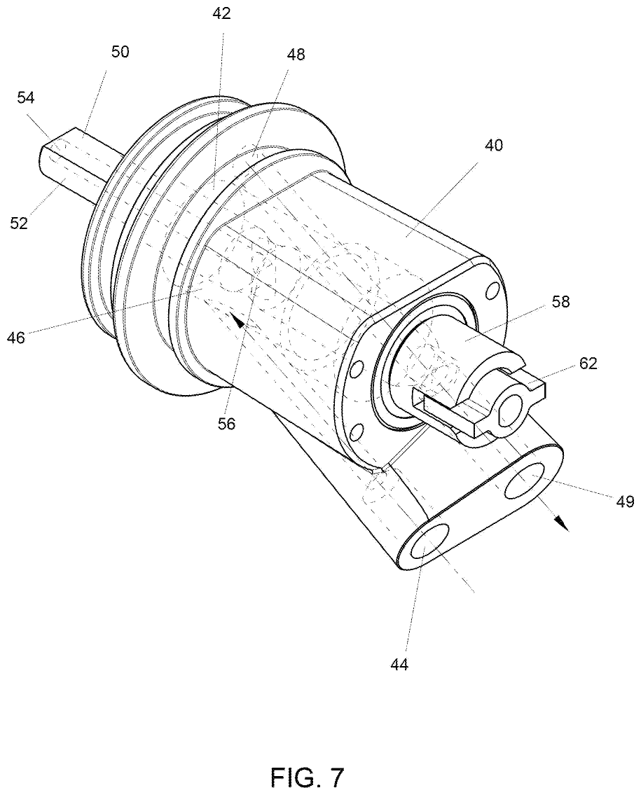

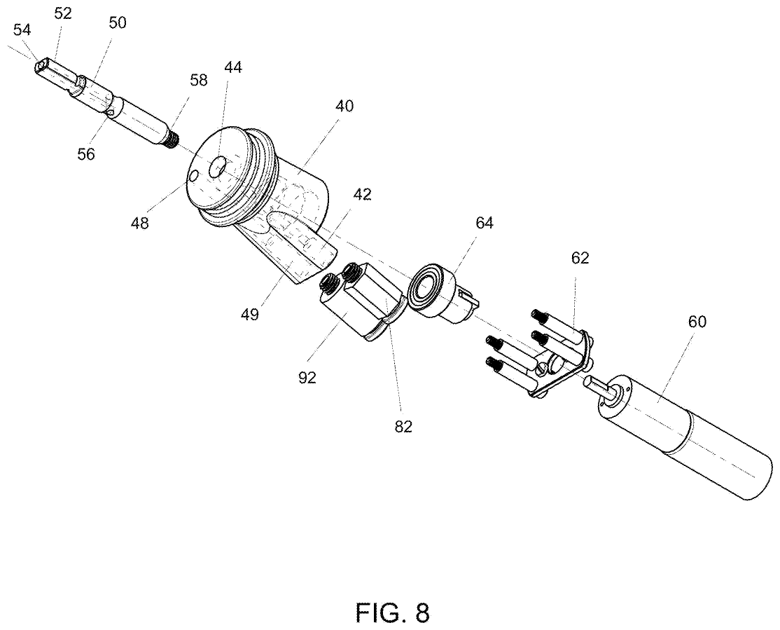

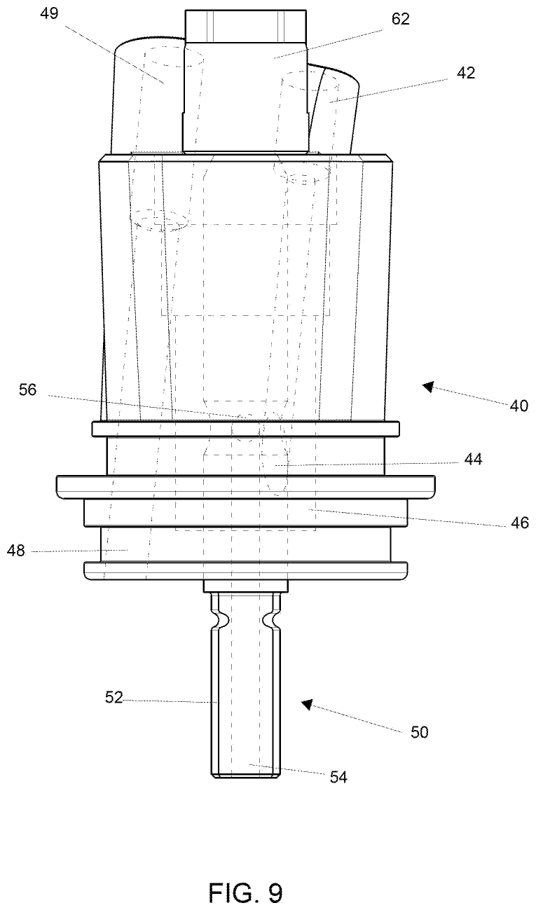

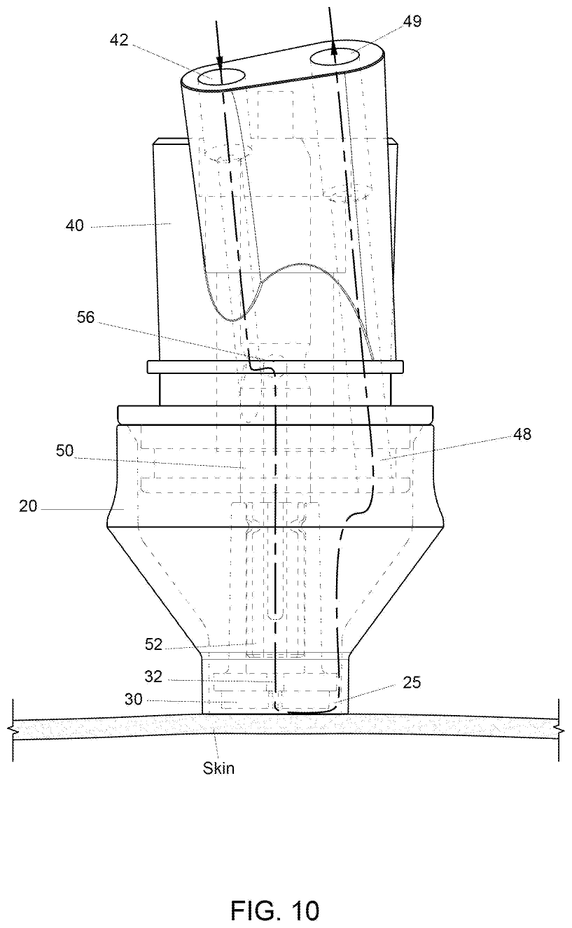

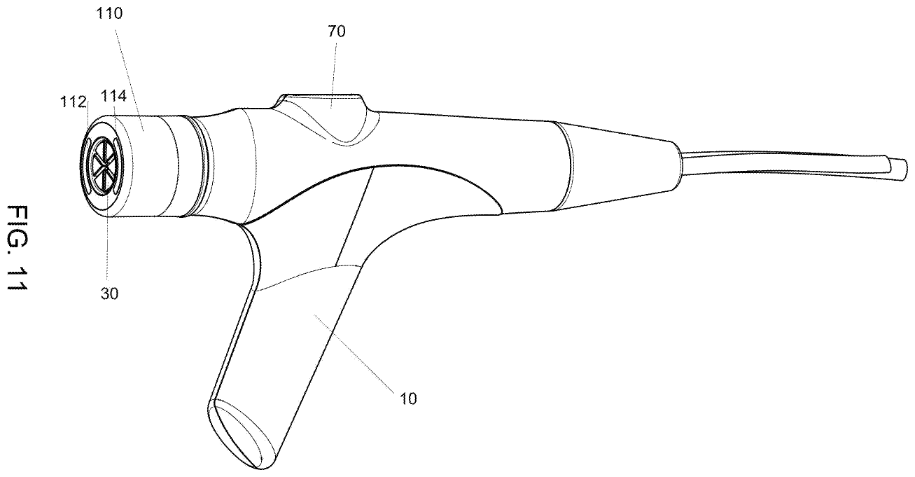

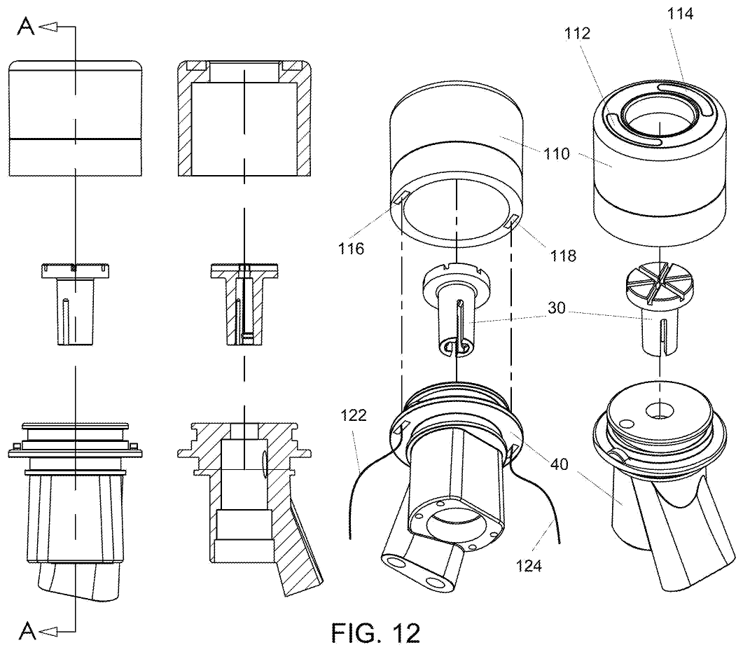

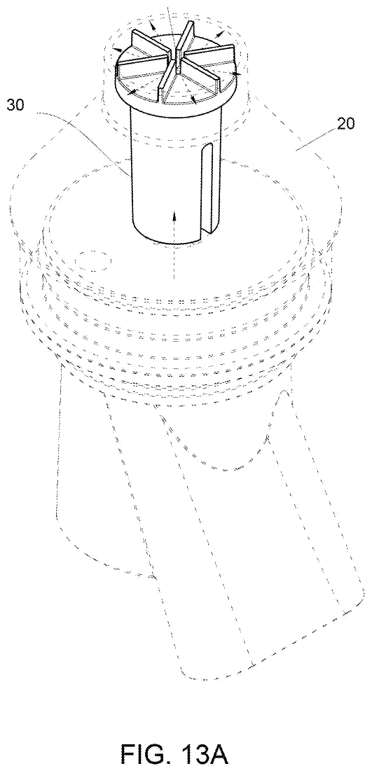

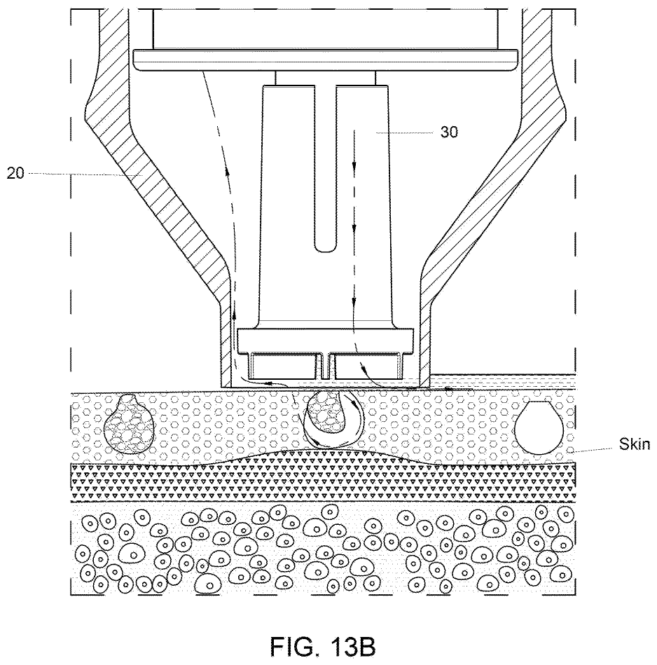

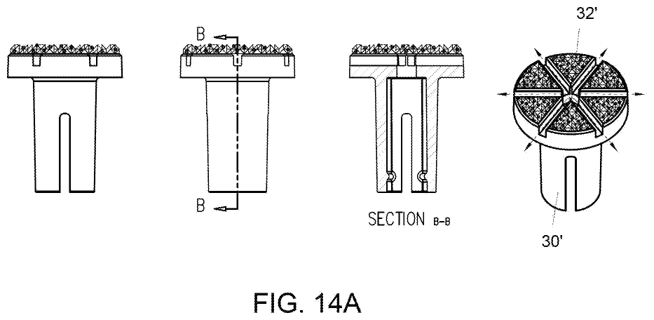

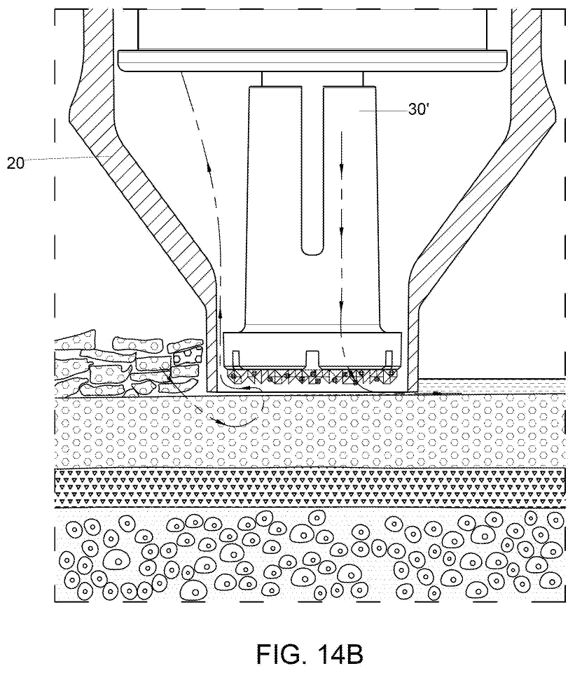

The following reference numerals identify components shown in FIGS. 1 through 14B:
10—housing.
20—tip cover.
25—gap between the treatment tip and internal walls of the tip cover.
30—treatment tip.
32—opening in treatment tip 30 or abrasive treatment tip 30′.
30′—abrasive treatment tip.

Reference numeral 32 identifies the same treatment-tip opening when treatment tip 30 is implemented as abrasive treatment tip 30′.
40—core.
42—main shaft opening in core 40.
44—core inlet.
46—cavity providing fluid communication between core 40 and shaft 50.
48—suction opening.
49—core outlet.
50—shaft.
52—shaft outlet at the proximal end of shaft 50.
54—distal end of shaft 50.
56—shaft aperture providing fluid communication between core 40 and shaft 50.

Reference numeral 56 identifies the shaft aperture that also permits treatment fluid to leave shaft 50.
60—electric motor.
62—motor mounting structure.
64—coupling between electric motor 60 and shaft 50.
70—user-operated switch.
80—treatment-fluid inlet tube.
82—first tube coupling connected to core inlet 44.
90—vacuum-return tube.
92—second tube coupling connected to core outlet 49.
100—treatment-fluid-container attachment structure.
102—treatment-fluid cartridge.
104—coupling structure for treatment-fluid cartridge 102.
110—electrode-carrying tip-cover embodiment.
112 and 114—electrodes that apply electrical energy to skin.
116 and 118—electrical connections for electrodes 112 and 114.
122 and 124—conductors extending through core 40 to electrical connections 116 and 118.

The devices and systems described herein can combine abrasion, electrical treatment, and treatment-fluid delivery at the working end. Electroporation, ultrasound, microcurrent, iontophoresis, radio-frequency energy, or another disclosed electrical or acoustic modality can be used when the corresponding electrode or transducer is present. Treatment fluid is supplied from a reservoir, and used treatment fluid is withdrawn through a vacuum-return path. A positive-pressure fluid source can be used in addition to or instead of a vacuum-induced supply flow.

A device for treating a skin surface includes a handpiece having a removable treatment tip at a distal end. The treatment tip can include one or more electrodes, an abrading end portion carrying an abrasive medium, one or more treatment-fluid outlets, and one or more vacuum inlets. The electrode structure, abrading structure, treatment-fluid-delivery structure, and vacuum-return structure can be incorporated into one removable tip or into separately removable structures.

The abrasive treatment tip can include an adhesive, such as epoxy resin, and an abrasive medium, such as emery particles.

An abrasive coating can be formed by electrostatically applying an adhesive to a treatment surface, electrostatically applying abrasive particles to the adhesive, and curing the adhesive at 80 degrees Celsius for two hours.

A treatment tip can combine a smooth skin-contact portion, an abrasive skin-contact portion, and molded grooves that maintain treatment-fluid flow from a treatment-fluid outlet to a vacuum inlet while removed material is withdrawn.

A motor-driven treatment tip is connected to a spindle shaft and rotates at a selected speed. In one operating range, the motor rotates the treatment tip from 10 to 300 revolutions per minute. The selected speed, abrasive structure, contact force, and treatment duration determine the amount of superficial material removed during a treatment pass.

A treatment tip can be formed from a polymer and can carry aluminum oxide, emery particles, metal particles, ceramic particles, or polymer particles bonded by an adhesive or polymer layer. One or more treatment-fluid openings provide a treatment-fluid outlet, a vacuum inlet, or both and maintain a fluid path across the skin-contact region.

Molded grooves and surface structures define treatment-fluid paths across the treatment face. The grooves keep portions of the treatment-fluid path open while distal portions of the treatment face contact skin, and suction moves treatment fluid and removed material toward the vacuum inlet.

A motor-driven treatment tip can remain at a selected treatment location while rotation moves the skin-contact structures across that location. This operating mode permits localized treatment without requiring repeated manual strokes over the same location.

For treatment of a body region, the operator can select a treatment-tip diameter, abrasive grade, rotational speed, treatment-fluid-delivery rate, and suction setting suited to the treatment region.

The handpiece housing can be shaped to support the operator's hand while the electric motor supplies treatment-tip movement. The operator can guide the handpiece in a continuous stroke or hold the handpiece at one treatment location without manually producing the local rotational movement.

In another embodiment, a detachable cap carries one or more electrodes for applying electrical stimulation while the treatment-fluid-delivery and vacuum-return structures operate.

The handpiece can include a treatment-fluid-container interface shaped to receive a bottle or cartridge. The interface can include threads, a puncturing member, an annular seal, or a combination of those structures. Insertion of the container opens a sealed treatment-fluid path from the container to a treatment-fluid conduit within the handpiece.

Treatment fluid passes through the treatment-fluid conduit, the spindle shaft, and the treatment tip to the skin-contact region. The treatment fluid can be supplied at a selected temperature and can contain cleaning, hydrating, cooling, warming, tightening, brightening, conductive, or other disclosed treatment agents.

A removable treatment-fluid container permits the operator to replace one treatment fluid with another without replacing a larger reservoir connected to the console.

The operator can select a treatment fluid according to the intended cleaning, hydration, electrical-conduction, temperature, or topical-delivery operation.

The treatment-fluid-container interface and a metered pump can limit delivery to a selected volume or selected flow rate for a treatment interval.

The removable container and sealed connection permit replacement of treatment fluid between treatment procedures while maintaining a closed supply path within the handpiece.

Microdermabrasion techniques that use stationary abrasive tips or manual movement can require repeated passes to treat a region. Repetitive linear motion can increase treatment time and can concentrate friction at portions of the skin surface.

A motorized microdermabrasion tip rotates while an abrasive treatment face contacts skin. The motor can operate from 1 revolution per minute to 2,000 revolutions per minute. The selected rotational speed determines the number of treatment-face contacts made during a treatment interval.

Rotation moves an abrasive treatment surface through a circular path and supplies local treatment directions without requiring the operator to create each direction by manual cross-hatching. The selected speed, contact force, abrasive grade, and treatment duration determine the amount of superficial exfoliation.

Rotation also produces repeated, controlled mechanical contact across the treatment region. The operator can select a speed and treatment-tip structure that distribute contact across the region while avoiding a repeated linear stroke over the same path.

Treatment fluid can be delivered while the treatment face rotates. The treatment fluid occupies recessed portions of the treatment face, reduces dry contact at those recessed portions, and carries removed material toward the vacuum inlet.

### Motorized Treatment Operations

The motorized treatment tip combines rotation of the treatment face, delivery of treatment fluid, and vacuum return. A controller or manual control can select a rotational speed up to 2,000 revolutions per minute to change the number of treatment-face contacts made during a treatment interval.

Unlike a stationary treatment face, the rotating treatment face moves ridges, channels, projecting members, or abrasive particles through multiple circumferential positions while the handpiece remains at one treatment location or moves along one treatment path.

In an embodiment that includes electroporation, selected electrical pulses are applied through electrodes to increase permeability of the stratum corneum during treatment-fluid delivery. The amount of transport depends on the pulse parameters, electrode geometry, treatment-fluid composition, delivered agent, and skin condition.

In one embodiment, an abrading surface, treatment-fluid delivery, electrical-current delivery, and vacuum return operate in a coordinated treatment sequence. The abrading surface removes superficial material from the stratum corneum. Treatment fluid is then delivered to the treated surface while electrical energy is applied through one or more electrodes. The vacuum-return path withdraws used treatment fluid and removed material. The sequence can be selected to resurface the outer skin surface and to increase movement of a treatment fluid through superficial pathways without requiring removal of dermal or hypodermal tissue.

The treatment-fluid supply and vacuum-return paths permit delivery and recovery of a low-viscosity treatment fluid. The abrasive surface can reduce superficial barrier material before or during treatment-fluid delivery. When an electrical modality is selected, the electrodes apply electrical energy while treatment fluid is present at the skin-contact region.

As used in this disclosure, the term ‘include’ and variations of that term are open-ended and do not exclude additional components or operations. The terms ‘comprise,’ ‘comprising,’ and ‘comprises’ have the same open-ended meaning.

In one embodiment, a skin-treatment device includes a handpiece, a removable treatment tip at a distal end of the handpiece, an electrical conductor, a treatment-fluid-delivery conduit, and a vacuum-return conduit. One or more user controls can control electrical energy, treatment-fluid delivery, suction, motor operation, or a combination of those functions. The controls can be located on the handpiece or on a connected console. The vacuum-return conduit carries used treatment fluid and removed material to a waste receptacle located in the handpiece or the console.

The handpiece can have a cylindrical, elliptical, square, rectangular, or contoured cross section and can include a molded hand grip. The handpiece can be formed from a medical-grade polymer, aluminum, stainless steel, another corrosion-resistant metal, or a combination of polymer and metal.

The handpiece includes an interior and an outer housing. A treatment-fluid-delivery conduit extends through the interior from a reservoir to one or more treatment-fluid outlets in the treatment tip. A vacuum-return conduit extends from one or more vacuum inlets in the treatment tip to a vacuum source and waste receptacle. One or more electrical conductors connect control circuitry with electrodes or an electric motor. In another embodiment, a portable handpiece contains a miniature vacuum source, a treatment-fluid reservoir, and a waste receptacle within the housing.

The treatment tip can be tapered, cylindrical, oval, square, rectangular, or contoured. A fluid-delivery portion can be domed or planar and can include one opening or multiple openings. Multiple openings distribute treatment fluid across the skin-contact region. The fluid-delivery portion can extend beyond, terminate flush with, or terminate behind the distal contact portions of the abrading structure.

A domed or planar contact portion supports the skin near a vacuum inlet and limits localized displacement of the skin into the vacuum inlet while suction is applied.

The vacuum inlet is positioned relative to the distal contact portions and treatment-fluid outlet to produce sufficient suction for return flow while limiting localized skin displacement. When the treatment tip contacts skin, the treatment-tip geometry defines a return-flow space between the treatment-fluid outlet and the vacuum inlet.

In one embodiment, the treatment-fluid outlet, abrading end portion, and electrodes terminate at approximately the same distal plane. This arrangement supports the skin while abrasion, treatment-fluid delivery, electrical-current delivery, and suction are applied and limits movement of the skin into a vacuum space.

In another embodiment, the vacuum inlet is positioned in a central, peripheral, annular, or circumferentially spaced portion of the treatment tip. The inlet position is selected to retain treatment fluid at the skin-contact region while still withdrawing used treatment fluid and removed material.

The abrading structure is positioned relative to the treatment tip so that its abrading end portion contacts the skin. The abrading end portion can terminate flush with, beyond, or behind another distal portion of the treatment tip.

The abrading structure can provide a nonabrasive smooth surface, a lightly abrasive surface, or a more abrasive surface according to the selected treatment. The abrading structure can surround the treatment-fluid outlet and vacuum inlet and can be positioned radially inside one or more electrodes. Other concentric or circumferential arrangements can also be used.

The abrading structure can be reusable or disposable in whole or in part. An integral abrading end portion can be cleaned and reused or discarded after use. An abrasive medium can also be carried on a removable strip or insert so that the underlying treatment-tip structure is reusable and the abrasive strip or insert is disposable.

The abrasive medium can include sandpaper, a medical-grade abrasive textile, a wire brush, carbon fibers, microneedles, aluminum oxide particles, polymer beads, ceramic particles, metal particles, carbon particles, conductive polymers, or conductive elastomers. A conductive abrasive medium can provide electrical contact, and a nonconductive abrasive medium can be electrically isolated from adjacent electrodes. A medical-grade textile can be formed from 100 percent medical-grade nylon and can carry one or more coatings or finishes.

The abrading end portion can have different thicknesses, diameters, and abrasive grades. Abrasive particles can be bonded to a polymer substrate, including an acrylonitrile-butadiene-styrene substrate. Illustrative abrasive grades range from 50 grit to 300 grit and can include aluminum oxide, sodium bicarbonate, or polymer particles. Coarser particles can be selected for an initial treatment or a body region, and finer particles can be selected for a later treatment or a more delicate region. An abrasive surface can also be formed by knurling, machining, laser treatment, molding, or chemical treatment. In one embodiment, the abrasive particles have a grade of 120 grit or coarser and an approximate diameter of 0.0044 inch.

The abrasive layer can have a thickness from 0.5 micrometer to 150 micrometers and, in one embodiment, from 15 micrometers to 120 micrometers. Individual abrasive particles or molded surface features can have lateral dimensions from 1 micrometer to 25 micrometers. The overall abrading end portion can have a diameter from 0.1 inch to 5 inches, corresponding to approximately 2.5 millimeters to 127 millimeters.

Electrical energy can be delivered from the device to the skin through one electrode or multiple electrodes. The electrodes can be formed as nodes, bars, rings, arcs, pads, or other conductive structures having dimensions selected for the intended treatment region.

Electrical or acoustic treatment modalities that can be incorporated into an embodiment include the following:

Electroporation uses electrical pulses to produce a temporary increase in membrane or skin-barrier permeability. In an electroporation embodiment, a pulse source applies selected pulses through electrodes while treatment fluid is present at the skin-contact region.

Microcurrent treatment applies a current from 10 microamperes to 500 microamperes through skin-contact electrodes. The selected current can remain below a level that produces a visible muscle contraction.

Iontophoresis applies an electrical potential through an electrode to move an ionized treatment agent having a charge opposite the charge of the electrode. The treatment agent can include a polar molecule, a hydrophilic molecule, a peptide, or another ionizable skin-treatment agent.

Sonophoresis applies ultrasonic mechanical energy through a transducer to increase movement of a topical composition through the epidermis and skin appendages. A gel, cream, ointment, or liquid can couple the ultrasonic transducer to the skin.

A galvanic-current embodiment applies direct electrical current through electrodes and can be used for iontophoresis.

An ultrasound embodiment applies cyclic acoustic pressure through an ultrasonic transducer. A frequency greater than approximately 20 kilohertz is above the upper limit of human hearing and can be selected according to the intended sonophoresis or acoustic-treatment operation.

An ultrasonic-cavitation embodiment can include selectable source frequencies of 3 megahertz, 1.4 megahertz, 1 megahertz, or 47 kilohertz. The selected frequency, power, duty cycle, coupling medium, transducer geometry, and treatment duration determine the acoustic field applied to the treatment region.

Acoustic cavitation can occur when alternating compression and rarefaction cycles in a liquid cause microscopic gas or vapor cavities to grow and collapse. An embodiment intended to produce acoustic cavitation includes a liquid coupling path and an ultrasonic transducer operated at selected power and frequency settings.

A treatment fluid used with the device can be selected for use before or after a separate mesotherapy procedure in which a practitioner injects a composition into tissue. The handpiece described herein does not require an injection needle unless a separate microneedling or injection structure is expressly included.

A radio-frequency embodiment applies radio-frequency electrical energy through one or more electrodes. The electrode geometry, frequency, power, duty cycle, contact time, and temperature limit are selected to control heating at the treatment region.

A heating or cooling embodiment includes a thermal element that changes the temperature of the treatment surface or treatment fluid. A source component can be adjustable within a range from 5 degrees Fahrenheit to 140 degrees Fahrenheit, and the operating setting is selected for the intended skin-contact procedure.

In one embodiment, a microcurrent waveform between 10 microamperes and 500 microamperes is delivered through one or more electrodes. The handpiece can remain at one location for a selected interval or can be moved in a continuous stroke while the current is applied.

In another embodiment, the device applies two or more electrical or acoustic modalities sequentially or concurrently. For example, an ultrasonic treatment can be followed by microcurrent treatment at the same region or at different regions. Each modality has a separately selected frequency, amplitude, duration, and electrode or transducer configuration.

Treatment fluid is delivered from a reservoir in the handpiece or in a connected console through a treatment-fluid conduit and one or more outlets in the treatment tip. The treatment fluid can clean the skin, carry a topical agent, provide electrical conductivity, or perform a combination of those functions. The treatment fluid can include water, a salt, an ionic surfactant, a nonionic surfactant, a preservative, an alcohol, glycerol, a gel-forming agent, or a combination of those ingredients. In one embodiment, a higher-conductivity treatment fluid has a conductivity from 1,000 microsiemens per centimeter to 100,000 microsiemens per centimeter. A lower-conductivity treatment fluid can have a conductivity from 0.1 microsiemens per centimeter to 999 microsiemens per centimeter. A selected conductive treatment fluid can have a conductivity from 500 microsiemens per centimeter to 50,000 microsiemens per centimeter.

A treatment fluid can include a skin-treatment liquid, lotion, vitamin composition, or pharmacologically active agent at a selected concentration. Disclosed agents include trichloroacetic acid, glycolic acid, an alpha-hydroxy acid, lactic acid, citric acid, phenol, botulinum toxin type A, phosphatidylcholine, aminophylline, hyaluronic acid, L-carnitine, vitamins, amino acids, collagen, lidocaine, heparin, elastin, glutathione, a hormone-replacement agent, hyaluronidase, a copper-manganese-zinc-sulfate-chromium composition, an ionic tissue-growth gel, an enzyme, a peptide, or a steroid. Use of a pharmacologically active agent is subject to the concentration, route of administration, and professional controls applicable to that agent.

A treatment fluid can also include a plant-derived ingredient, fruit-derived ingredient, enzyme, stem-cell-derived ingredient, healing agent, anti-inflammatory agent, antifungal agent, antiviral agent, antibacterial agent, or acaricidal agent. Microdermabrasion removes superficial material from the skin surface, and an accompanying treatment fluid can be selected to support the intended post-abrasion treatment.

Technical background concerning skin electroporation is described by Anne-Rose Denet, Rita Vanbever, and Veronique Preat, ‘Skin Electroporation for Transdermal and Topical Delivery,’ Advanced Drug Delivery Reviews, volume 56, issue 5, pages 659-674 (2004).

A vacuum source can apply suction through the vacuum-return conduit and vacuum inlet. One illustrative vacuum pump has a current rating of 2.9 amperes, a maximum flow rate of 2 cubic feet per minute, a power rating of 120 watts, and an electrical frequency of 60 hertz, and can comply with the Restriction of Hazardous Substances requirements. Other pump ratings can be selected according to the conduit dimensions, treatment-tip geometry, and intended suction range. A flow-control valve or variable-speed motor controller can increase or decrease suction.

In one embodiment, a handpiece receives removable and interchangeable treatment-tip structures. An outer structure carries one or more electrodes and associated conductors. An intermediate structure carries an abrading end portion. An inner structure includes one or more treatment-fluid outlets and one or more vacuum inlets.

The inner structure is located near the center of the treatment tip, the outer structure is located near the periphery, and the intermediate structure is located between them. The inner, intermediate, and outer structures can be coaxial and can have circular or noncircular annular shapes. The abrading end portion on the intermediate structure can surround the treatment-fluid outlet and vacuum inlet, and the electrodes on the outer structure can surround the abrading end portion.

The inner, intermediate, and outer structures can be connected to the handpiece by compression fittings, threaded fittings, bayonet fittings, snap fittings, or keyed mechanical fittings. One or more structures can be formed from stainless steel or a medical-grade polymer. The intermediate structure can be a reusable stainless-steel structure carrying diamond abrasive particles or a disposable polymer structure carrying a disposable abrasive medium. The inner structure can be transparent, detachable, disposable, or a combination of those properties. In another arrangement, an electrode is positioned near the center, a treatment-fluid structure surrounds the electrode, and an abrasive structure forms an outer region.

In another abrading structure, the abrading end portion includes one or more rounded, straight, curved, radial, circumferential, or intersecting grooves. The grooves maintain a fluid path, guide removed material toward the vacuum inlet, and contact skin at the sides of a follicular opening. Groove dimensions and patterns can be selected for different treatment-tip diameters and treatment regions.

### Motor-Current and Rotational-Speed Control Embodiments

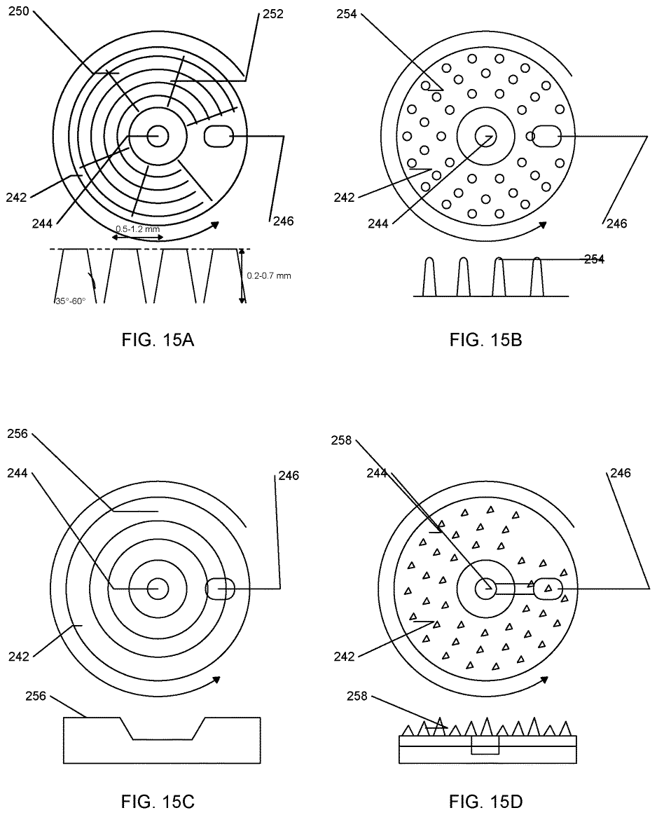

A hydrodermabrasion system 200 controlled with measured motor-current values and measured rotational-speed values is described with reference to FIGS. 15A through 15D, FIGS. 16A through 16C, and FIG. 17. The system 200 may incorporate any of the handpiece, treatment-tip, abrasive, electrode, treatment-fluid-delivery, vacuum-return, treatment-fluid-container, and stationary-treatment structures described above. The reference numerals used for system 200 identify an additional embodiment and do not restrict use of the previously described components.

The processor 212 receives motor-current measurements and rotational-speed measurements and sends rotational-speed commands, treatment-fluid-delivery commands, and suction commands to the motor driver 268, pump driver 274, and vacuum-control actuator 280. Those components change operation of the electric motor 218, variable-rate treatment-fluid pump 206, and vacuum source 204 in response to the commands.

The motor-current and rotational-speed control may operate without electrical stimulation. The electrodes 248, electroporation components, ultrasound components, microcurrent components, and therapeutic agents described for other embodiments are optional and are not required for motor-current sensing, rotational-speed sensing, motor-speed control, treatment-fluid delivery, suction control, or vacuum return.

The system 200 includes a console 202 having a vacuum source 204, a variable-rate treatment-fluid pump 206, a treatment-fluid reservoir 208, a waste receptacle 210, and a processor 212. The vacuum source 204 can provide continuous or variable suction. The treatment-fluid pump 206 can be a peristaltic pump, diaphragm pump, piston pump, or gear pump capable of changing a treatment-fluid-delivery rate in response to a command from the processor 212.

The system 200 further includes a memory 272, a motor driver 268, a pump driver 274, a user control 276, a vacuum-control actuator 280, and a status indicator 282. The motor driver 268 changes electrical power supplied to the electric motor 218 in response to a rotational-speed command. The pump driver 274 changes electrical power or mechanical drive supplied to the variable-rate treatment-fluid pump 206 in response to a treatment-fluid-delivery command.

The vacuum-control actuator 280 changes suction produced at the vacuum inlet 246 in response to a suction command from the processor 212. The vacuum-control actuator 280 may include a variable-speed motor controller for the vacuum source 204, a proportional valve in the vacuum-return path, an electrically operated pressure regulator, or a series combination of those components. The status indicator 282 may include a visible light source, a display, an audible sound generator, or a combination of those components.

The treatment-fluid reservoir 208 is connected to an inlet of the variable-rate treatment-fluid pump 206. The waste receptacle 210 is connected to the vacuum-return path downstream from the vacuum inlet 246. A supply check valve may prevent reverse flow toward the treatment-fluid reservoir 208, and a return check valve may prevent material in the waste receptacle 210 from moving toward the handpiece 216.

A multi-lumen conduit 214 couples the console 202 to a handpiece 216. The conduit 214 may include a treatment-fluid supply lumen, a vacuum-return lumen, one or more electrical conductors, and one or more auxiliary lumens or conductors. The lumens may be formed in a common extrusion, carried in a sleeve, or provided as separate tubes.

The handpiece 216 includes an electric motor 218 and a spindle or other rotatable drive member 220. A removable treatment tip 222 is mechanically coupled to the drive member 220 so that operation of the electric motor 218 rotates or oscillates the treatment tip 222. The treatment tip 222 can be coupled by threads, a bayonet connection, a snap fit, a friction fit, or a keyed coupling.

The handpiece 216 includes an elongate handpiece housing 217 extending from a proximal connector 224 to a distal opening 226. A fluid-tight wall 229 within the handpiece housing 217 bounds a motor compartment 228. The electric motor 218 and motor driver 268 are positioned within the motor compartment 228, and the rotatable drive member 220 extends from the motor compartment 228 toward the distal opening 226.

An electrical connector 288 at the proximal connector 224 includes a first conductor that carries motor-current measurements from the motor-current sensor 270, a second conductor that carries rotational-speed measurements from the rotational-speed sensor 278, a third conductor that carries motor commands to the motor driver 268, a fourth conductor that supplies electrical power to the motor driver 268 and the sensors 270 and 278, and a fifth conductor that provides an electrical return path. The conductors can be separate wires, conductive traces on a flexible circuit, or contacts in a multi-contact connector.

The handpiece 216 can include a treatment-fluid-container interface 230 configured to receive a treatment-fluid container 231 in the form of a bottle, cartridge, or ampule. The interface 230 can include a threaded connector, a puncturing member 290, an annular container seal 292, and a valve. When the longitudinal axis of the handpiece 216 is horizontal, a longitudinal axis of the treatment-fluid container 231 can form an acute angle with the longitudinal handpiece axis and can descend toward the distal opening 226. The treatment-fluid container 231 can be positioned distal to the electric motor 218 to assist priming and distribute handpiece mass.

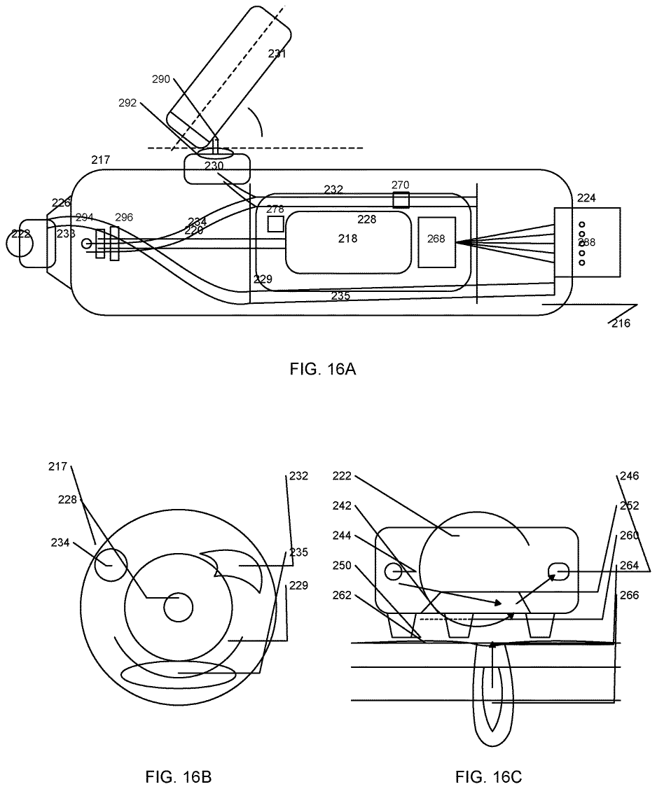

As shown in FIG. 16A, a first treatment-fluid conduit 232 extends from the treatment-fluid-container interface 230 beside the motor compartment 228, and a bypass conduit 234 extends from the first treatment-fluid conduit 232 to a core inlet 233 at the distal opening 226. As shown in FIG. 16B, the first treatment-fluid conduit 232 has a noncircular cross section bounded by a concave inner wall facing and partially surrounding a circumferential portion of the motor compartment 228 and a convex outer wall facing the exterior wall of the handpiece housing 217. A vacuum-return passage 235 extends from the distal opening 226 toward the proximal connector 224 and is separated from the treatment-fluid conduits and the motor compartment 228 by the fluid-tight wall 229.

A treatment-fluid supply seal 294 surrounds the connection between the bypass conduit 234 and the core inlet 233. A second annular seal separates the treatment-fluid supply path from the vacuum-return passage 235. A rotary seal 296 or a stationary seal surrounding the drive member 220 separates the motor compartment 228 from treatment fluid and material carried by the vacuum-return stream. Each seal contacts facing physical surfaces and closes a leakage path between those surfaces.

The noncircular cross section of the first treatment-fluid conduit 232 is defined by the concave inner wall, the convex outer wall, and two connecting sidewalls. The concave inner wall follows part of the circumference of the motor compartment 228 and provides a treatment-fluid passage beside the motor compartment without placing treatment fluid inside the motor compartment.

The treatment tip 222 may include an inner structure 236, an intermediate annular structure 238, and an outer structure 240 arranged coaxially. The inner structure 236 may include a fluid outlet 244 and a vacuum inlet 246. The intermediate annular structure 238 carries an active skin-contact face 242. The outer structure 240 may carry one or more electrodes 248 and conductors for applying electrical stimulation to a treatment region. The fluid outlet 244 and the vacuum inlet 246 may be concentric, circumferentially spaced, radially spaced, or arranged in another pattern that produces a fluid path across the skin-contact face 242.

The expression “skin-contact face” identifies the distal treatment surface 242. A distal contact portion is a ridge end, a projecting-pin end, a projecting-nodule end, an abrasive particle, or a planar contact land that can touch the skin surface 262. A recessed passage is a fluid-flow space bounded by a passage floor and sidewalls, by an annular recess and adjacent contact portions, or by spaces between projecting members.

When the distal contact portions touch the skin surface 262, the floor of the recessed passage remains spaced from the skin surface. Treatment fluid can therefore occupy a nonzero cross-sectional flow area from the fluid outlet 244 to the vacuum inlet 246 while the treatment tip 222 contacts the skin surface 262.

The inner structure 236, the intermediate annular structure 238, and the outer structure 240 may be molded or machined as one piece or assembled from separate components. One or more of the structures may be transparent or translucent to permit observation of fluid and debris. Suitable materials include medical-grade polymers, stainless steel, ceramic materials, elastomers, and combinations thereof. A portion that contacts the skin may be disposable while another portion is reusable.

FIG. 15A illustrates a grooved skin-contact face 242 having raised ridges 250 separated by recessed channels 252. An inclined sidewall of each raised ridge 250 can form an angle from 35 degrees to 60 degrees relative to a plane containing distal ends of the raised ridges 250. Each recessed channel 252 can have a depth measured from that plane to a floor of the recessed channel from 0.2 millimeter to 0.7 millimeter, and adjacent raised ridges 250 can have a center-to-center spacing from 0.5 millimeter to 1.2 millimeters. The sidewall angle, channel depth, and center-to-center spacing can be selected independently and can vary across the skin-contact face 242.

The channels 252 maintain one or more treatment-fluid paths when the skin-contact face 242 contacts compliant skin. During rotation, the ridges 250 move through different circumferential positions and direct portions of the treatment fluid through the channels 252 toward the vacuum inlet 246. Contact between successive ridges and skin beside a follicular opening may alternately compress and release the contacted skin region.

In the grooved embodiment, at least one recessed channel 252 extends along a first direction from the outlet region toward the inlet region, and at least one other recessed channel 252 extends along a second direction different from the first direction. The different channel directions redirect treatment fluid through different paths while the ridges 250 rotate through successive positions over the treatment region.

FIG. 15B illustrates an alternative skin-contact face 242 having projecting pins or nodules 254. The pins or nodules 254 may be rounded, conical, frustoconical, cylindrical, faceted, or irregular. They may be arranged in concentric rows, radial rows, a staggered array, a random distribution, or another pattern. A distal end of each pin or nodule may be smooth, textured, resilient, or abrasively coated.

Each projecting pin or projecting nodule 254 has a base joined to a substrate of the intermediate annular structure 238 and a distal contact end spaced from that base. The projecting members may be arranged in circumferentially offset rows so that a projecting member in one row passes a skin location at a different time from a projecting member in an adjacent row. Spaces between the bases and distal contact ends remain connected to the fluid outlet 244 and the vacuum inlet 246.

FIG. 15C illustrates a planar skin-contact surface 256. A continuous planar contact land surrounds or adjoins an annular recessed passage. Fluid flow is maintained by the relative locations of the fluid outlet 244 and vacuum inlet 246 and by the annular recessed passage that remains open while the planar contact land touches skin.

The planar treatment face 256 includes a continuous planar contact land and an annular recessed passage positioned radially inside or radially outside the planar contact land. The fluid outlet 244 opens into a first portion of the annular recessed passage, and the vacuum inlet 246 opens into a second portion spaced from the first portion, thereby establishing a physical path across the planar treatment face 256.

FIG. 15D illustrates an abrasive skin-contact surface 258. The abrasive surface 258 can include emery particles, aluminum oxide particles, diamond particles, metal particles, ceramic particles, silicon-carbide particles, or polymer particles bonded to a substrate by epoxy resin, another adhesive layer, a polymer layer, sintering, or plating. The electrostatic application and curing operations described above can be used to form the abrasive surface 258.

The abrasive treatment face 258 includes a substrate, an adhesive layer attached to the substrate, and abrasive particles partially embedded in the adhesive layer. Exposed portions of the abrasive particles provide distal contact portions. Spaces between the abrasive particles and at least one molded channel provide a fluid-flow path from the fluid outlet 244 to the vacuum inlet 246.

The treatment surfaces illustrated in FIGS. 15A through 15D may be used separately. FIG. 15A shows raised ridges and recessed channels. FIG. 15B shows projecting pins or nodules. FIG. 15C shows a planar treatment face with a surrounding recess. FIG. 15D shows abrasive particles blonded to a substrate. A treatment face may also contain two or more of these physical structures.

During treatment, the pump 206 supplies treatment fluid through the handpiece 216 and the fluid outlet 244. The vacuum source 204 draws the treatment fluid across at least part of the skin-contact face 242 and into the vacuum inlet 246. The withdrawn stream carries used treatment fluid, exfoliated skin material, sebum, and particulate material released from the skin to the waste receptacle 210.

As shown in FIG. 16C, treatment fluid leaves the fluid outlet 244, enters the recessed channel 252, an annular recess, or a peripheral clearance, moves across the skin surface 262, and enters the vacuum inlet 246. The treatment fluid occupies the recessed portions between the skin-contact face 242 and the skin surface 262. Ridges, pins, nodules, abrasive particles, or planar contact lands may contact the skin while treatment fluid moves through the adjacent recessed portions toward the vacuum inlet 246.

Rotation of the skin-contact face 242 mechanically shears treatment fluid together with native sebum and other hydrophobic material at the skin surface 262. This mechanical action can disperse droplets, particles, or films of the hydrophobic material into the treatment fluid. Mechanical emulsification in this disclosure means mechanically induced dispersion and does not require formation of a thermodynamically stable emulsion.

In one operating sequence, the variable-rate treatment-fluid pump 206 supplies treatment fluid before or while rotation begins. Treatment fluid occupies the recessed passage while successive distal contact portions move across the skin surface 262. The moving contact portions and moving treatment fluid separate a portion of native surface lipid from the skin and divide the separated lipid into droplets, films, or particles carried in the treatment fluid. The vacuum-return path withdraws the treatment fluid, separated lipid, and exfoliated material through the vacuum inlet 246.

With a grooved or projecting-member treatment face, a first distal contact portion can press skin on a first side of a follicular opening 264, and a later distal contact portion can press skin on a different side of the follicular opening 264 as the treatment tip 222 rotates. The skin can relax between successive contacts. Treatment fluid and suction move material released from the follicular opening 264 toward the vacuum inlet 246.

The fluid movement and rotation can increase wetting of the treatment region by an aqueous treatment fluid. Rotation can also reduce cohesion of surface material by agitation, dilution, dispersion, or a combination of those effects and can facilitate movement of the material toward the vacuum inlet 246.

A point on the rotating skin-contact face 242 moves tangentially relative to the skin. Grooves, ridges, pins, nodules, abrasive particles, and other local surface features produce additional local directions of fluid and tissue movement. The resulting circumferential, radial, and intersecting shear directions may produce sequential compression and relaxation at tissue margins around a follicular opening 264 and may assist movement of a sebaceous plug or other follicular material 266 toward the surface. Vacuum and fluid flow then carry released material toward the vacuum inlet 246.

The system 200 includes a motor-current sensor 270 connected in the electrical supply circuit of the electric motor 218 and a rotational-speed sensor 278 positioned to measure rotation of the drive member 220. The motor-current sensor 270 can include a current-sense resistor and a voltage-measurement circuit or a Hall-effect current sensor. The rotational-speed sensor 278 can include an optical encoder, a magnetic encoder, a Hall-effect rotary sensor, an optical interrupter, or a back-electromotive-force measurement circuit.

The motor-current sensor 270 measures electrical current rather than mechanical torque. When motor-driver voltage, commutation state, and commanded speed are known, a change in measured motor current provides an electrical indication of a change in mechanical load on the electric motor 218. The measured rotational speed supplies a separate physical measurement that distinguishes a load increase accompanied by slowing from a current change that occurs without slowing.

In a current-sense-resistor implementation, a signal-conditioning circuit 284 measures a voltage across a resistor having a known resistance, and an analog-to-digital converter 286 supplies a digital value representing that voltage to the processor 212. The processor 212 calculates motor current in amperes from the measured voltage and the known resistance. In a Hall-effect implementation, the analog-to-digital converter 286 converts the Hall-effect sensor output into a digital motor-current sample.

The rotational-speed sensor 278 may generate a stored number of pulses for each revolution of the drive member 220. The processor 212 counts pulses during a measured time interval or measures elapsed time between successive pulses and converts the result to revolutions per minute. Each motor-current sample and rotational-speed measurement may be associated with a clock value maintained by the processor 212.

The processor 212 can receive motor-current samples at a rate greater than 100 samples per second and can receive rotational-speed measurements at a selected measurement rate. The processor can calculate an arithmetic average or weighted average from a stored number of successive motor-current samples and can compare the calculated motor-current value and a measured rotational-speed value with stored numerical thresholds.

A moving-average filter may use four to sixty-four successive motor-current samples and may be updated after each new motor-current sample. A weighted filter may assign greater numerical weight to more recent samples. The stored number of samples and the numerical weights are fixed in the memory 272 before a treatment interval or selected from a stored table associated with the installed treatment tip 222.

The motor and motor driver may have a hardware operating range from 1 revolution per minute to 2,000 revolutions per minute. A treatment program may permit commands from 10 revolutions per minute to 2,000 revolutions per minute, and a lower-speed treatment program may limit commands to a range from 10 revolutions per minute to 300 revolutions per minute.

When a motor-current increase exceeds a stored positive motor-current-increase threshold and a rotational-speed decrease exceeds a stored positive rotational-speed-decrease threshold, the processor 212 may send a lower rotational-speed command to the motor driver 268 and a higher treatment-fluid-delivery command to the pump driver 274. The processor may retain the changed commands for a stored response interval and then compare a later filtered motor-current value with the filtered motor-current value measured before the command change.

The processor 212 can also control suction, electrical stimulation, treatment duration, a visible indicator, or an audible indicator. When a filtered motor-current value reaches the stored motor-stop threshold, or when measured rotational speed falls below a stored minimum rotational speed, the processor can stop the electric motor 218, send a suction command that produces less suction, continue treatment-fluid delivery for the release interval, and activate the visible indicator or audible indicator.

A treatment-fluid-delivery command may specify a pump rotational speed, a pump stroke frequency, a pump duty cycle, or a treatment-fluid delivery rate expressed in milliliters per minute. The memory 272 may store a calibration table that associates each pump command with a measured delivery rate for the variable-rate treatment-fluid pump 206 and connected fluid path.

A suction command may specify a vacuum-source motor speed, a proportional-valve position, a regulator position, a pressure measured relative to ambient pressure, or a return-flow rate. The release-suction command produces less suction at the vacuum inlet 246 than the selected suction command by reducing vacuum-source speed, opening a proportional valve, changing a regulator position, or a combination of those actions.

The handpiece 216 can be moved in a continuous linear treatment stroke while the treatment tip 222 rotates. Rotation supplies local circumferential movement, so the operator is not required to create every treatment direction by manually cross-hatching the handpiece. The handpiece can alternatively be held at a stationary location while the rotating treatment tip produces local exfoliation, treatment-fluid movement, and vacuum-assisted removal.

Electrical stimulation may be applied through the electrodes 248 before, during, or after rotation and fluid delivery. The electrodes 248 may be arranged in one or more rings, arcs, discrete pads, or other patterns. The treatment fluid may be selected to provide a desired electrical conductivity and may contain one or more skin-enhancing, cleaning, hydrating, cooling, heating, tightening, brightening, therapeutic, or conductive agents described above.

The treatment tip 222 may be supplied as a sterile or clean single-use component. A disposable tip may reduce transfer of biological material between patients and may include a pre-applied abrasive, a molded surface profile, a fluid passage, a vacuum passage, an electrode, or a combination thereof. Reusable components may be configured for cleaning, disinfection, or sterilization.

The dimensions, materials, speeds, treatment-fluid-delivery rates, suction levels, conductivities, and treatment agents described herein can be selected and combined when the resulting structures and operating settings are physically compatible.

References to treatment of multiple skin layers describe selectable treatment conditions and delivery pathways. A noninvasive surface-treatment embodiment removes material from the stratum corneum and does not require removal of dermal or hypodermal tissue. Delivery of a treatment agent beyond the stratum corneum depends on the selected agent, treatment surface, electrical modality when present, treatment duration, and skin condition.

### Specific Control and Fluid-Flow Structures

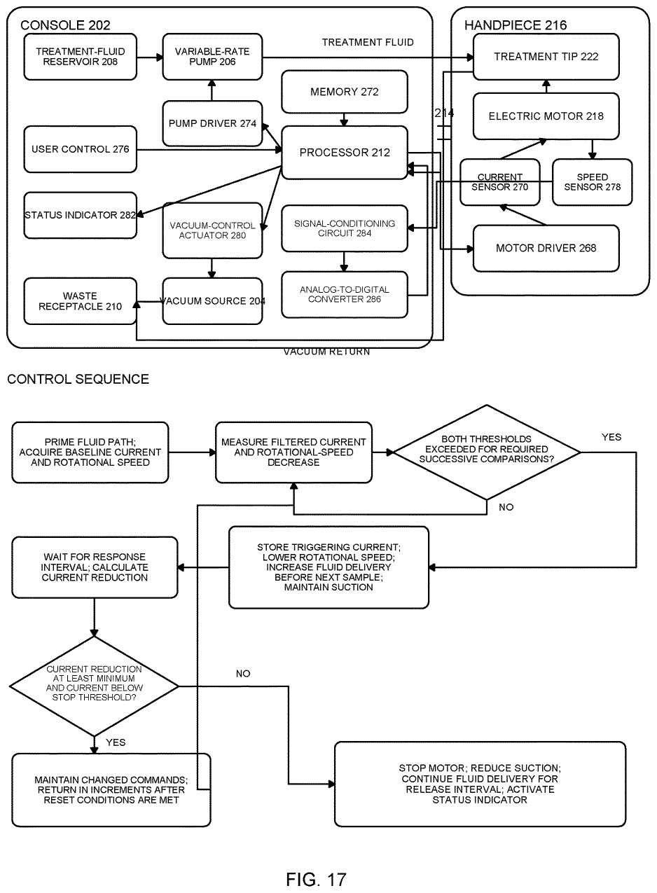

As shown in FIG. 17, the electronic controller includes the processor 212 and memory 272. The handpiece motor is connected to a motor driver 268. A motor-current sensor 270 is electrically connected in the motor supply circuit and sends measured motor-current values to the processor 212. A pump driver 274 receives pump commands from the processor 212 and supplies operating power to the variable-rate treatment-fluid pump 206. A user control 276 supplies a selected treatment mode, selected motor speed, selected fluid-delivery rate, and selected vacuum level to the processor 212.

The motor-current sensor 270 can include a current-sense resistor and signal-conditioning circuit 284 or a Hall-effect current sensor. An analog-to-digital converter 286 converts the sensor output into successive numerical motor-current values. The rotational-speed sensor 278 can include an encoder, Hall-effect position sensor, optical sensor, or back-electromotive-force measurement circuit and can provide measured rotational-speed values to the processor 212.

Before skin contact, the processor 212 may operate the pump 206 until treatment fluid reaches the fluid outlet 244 and may operate the vacuum source 204 until fluid reaches the waste receptacle 210. The processor 212 then commands the motor driver 268 to rotate the treatment tip 222 at the selected motor speed and commands the pump driver 274 to deliver treatment fluid at the selected fluid-delivery rate.

The memory 272 stores a first motor-current threshold, a reset motor-current threshold lower than the first motor-current threshold, a second motor-current threshold greater than the first motor-current threshold, a minimum motor-speed command, and a maximum pump-delivery command. The thresholds and commands may be stored for each treatment mode or treatment-tip type and may be changed through the user control 276.

The processor 212 receives more than 100 motor-current samples per second and calculates a filtered motor-current value from a predetermined number of successive samples. The filter can calculate an arithmetic average or a weighted average. The processor 212 compares each filtered motor-current value with the stored thresholds.

When the filtered motor-current value exceeds the first motor-current threshold, the processor 212 sends a lower motor-speed command to the motor driver 268 and a higher pump-delivery command to the pump driver 274. The processor 212 may repeat the speed reduction and delivery increase in stored increments until the filtered motor-current value falls below the reset motor-current threshold, the minimum motor-speed command is reached, or the maximum pump-delivery command is reached.

When the filtered motor-current value remains below the reset motor-current threshold for a stored number of successive samples, the processor 212 increases the motor-speed command and decreases the pump-delivery command toward the selected motor speed and selected fluid-delivery rate. Use of the first motor-current threshold and the lower reset motor-current threshold prevents repeated switching caused by small changes in measured motor current.

When the filtered motor-current value exceeds the second motor-current threshold, the processor 212 stops the motor through the motor driver 268. The processor 212 may also reduce vacuum, continue treatment-fluid delivery for a stored interval, and activate a visible or audible indicator. These actions permit release of skin from the treatment tip and replenishment of fluid at the treatment region.

The processor 212 writes the lower rotational-speed command to the motor driver 268 and the higher treatment-fluid-delivery command to the pump driver 274 before the next scheduled motor-current sample. The response interval begins after the processor has issued both commands. When the drivers provide command-acknowledgement signals, the response interval may begin after both acknowledgement signals are received.

If the processor 212 does not receive a valid rotational-speed signal, receives a motor-current value outside the measurement range, or does not receive a command-acknowledgement signal within a stored interval, the processor may stop the electric motor 218, send the release-suction command to the vacuum-control actuator 280, continue treatment-fluid delivery for the release interval, and activate the status indicator 282.

The treatment-fluid flow path extends from the treatment-fluid reservoir 208 through the variable-rate pump 206, the supply lumen of the multi-lumen conduit 214, the handpiece supply passage, and the fluid outlet 244; across the skin-contact face 242; and through the vacuum inlet 246, the handpiece vacuum-return passage, the return lumen of the multi-lumen conduit 214, and the waste receptacle 210. The vacuum source 204 applies negative pressure to the return side of this path.

The fluid outlet 244 and vacuum inlet 246 are positioned on different portions of the treatment tip 222. A recessed channel 252, an annular recess, or a peripheral clearance connects the outlet region with the inlet region. Treatment fluid therefore has a physical passage across the skin-contact face 242 when the distal contact portions of the treatment tip touch the skin surface 262.

The fluid layer 260 is the treatment fluid occupying the recessed channel 252, annular recess, peripheral clearance, and adjacent space between the skin-contact face 242 and the skin surface 262. The fluid layer 260 does not require complete separation of every portion of the skin-contact face from the skin. Direct contact may occur at ridges, pins, nodules, abrasive particles, or planar contact lands while fluid remains in the recessed portions.

For the grooved treatment face shown in FIG. 15A, rotation moves treatment fluid circumferentially along the ridges 250 and radially through the recessed channels 252. For the pinned or nodular treatment face shown in FIG. 15B, spaces between adjacent pins or nodules 254 provide fluid passages. For the planar treatment face shown in FIG. 15C, an annular recess or peripheral clearance provides the fluid passage. For the abrasive treatment face shown in FIG. 15D, spaces between abrasive particles and one or more molded channels provide the fluid passage.

The planar treatment face 256 includes a continuous planar skin-contact land that lacks projecting abrasive particles, raised ridges, pins, and nodules. A central outlet and a radially spaced vacuum inlet, or a radially spaced outlet and a central vacuum inlet, establish treatment-fluid movement through an annular recessed passage adjacent to the planar contact land.

Rotation of the treatment tip moves treatment fluid tangentially relative to the skin surface. Radial channels, circumferential channels, intersecting channels, pins, nodules, and abrasive particles redirect portions of the treatment fluid in additional directions. The moving treatment fluid mixes with sebum and other material removed from the skin surface, and the vacuum-return path carries the resulting mixture to the waste receptacle 210.

FIG. 16C shows the treatment tip 222 in contact with the skin surface 262, the fluid outlet 244, the fluid layer 260, the vacuum inlet 246, the follicular opening 264, and follicular material 266. Direction arrows show rotation of the treatment tip, movement of treatment fluid from the fluid outlet toward the vacuum inlet, and movement of removed material into the vacuum inlet. The depicted fluid layer is the physical volume of treatment fluid within the recessed flow path and adjacent clearance and does not imply complete separation of the treatment tip from the skin.

FIGS. 15A through 15D are arranged on one drawing sheet and depict the grooved, pinned or nodular, planar, and abrasive treatment faces and their treatment-fluid passages. FIGS. 16A through 16C are arranged on a second drawing sheet and depict the handpiece passages, motor isolation, treatment-fluid-container interface, motor driver, sensor locations, electrical connector, seals, and treatment-fluid path at the skin. FIG. 17 is arranged on a third drawing sheet and depicts the sensor circuits, processor, memory, motor driver, pump driver, vacuum control, numerical comparisons, command paths, response verification, and release sequence.

The three drawing sheets collectively illustrate the treatment-face structures, handpiece structures, fluid paths, electrical connections, measured values, command changes, response interval, motor-stop operation, suction reduction, and treatment-fluid release described below.

A treatment method can combine motorized movement of a treatment tip, treatment-fluid delivery, and vacuum-assisted removal during one treatment interval.

In one method, a motorized treatment tip exfoliates a treatment region while delivering treatment fluid.

The operator positions the motorized treatment tip against a selected treatment region.

The electric motor rotates or oscillates the treatment tip to move the skin-contact structures across the selected treatment region while the handpiece remains at one location or moves along a selected path.

A treatment-fluid conduit integrated with the treatment tip or positioned adjacent to the treatment tip delivers treatment fluid to the selected treatment region.

A vacuum-return path withdraws used treatment fluid and removed material while the treatment tip rotates or oscillates.

Exfoliation and treatment-fluid delivery can occur during the same treatment interval so that treatment fluid contacts the skin surface immediately after superficial material is removed.

The motor can rotate the treatment tip from 10 revolutions per minute to 2,000 revolutions per minute and can supply local treatment movement without requiring the operator to produce that movement manually.

In another method, the system performs localized microdermabrasion at a selected treatment location.

The operator places the motorized treatment tip against the selected treatment location without requiring a continuous linear handpiece stroke.

The electric motor moves the treatment-face structures across the selected treatment location while treatment fluid is delivered.

The vacuum-return path withdraws treatment fluid and exfoliated material from the selected treatment location.

The operator can maintain the handpiece at the selected treatment location for a selected interval while controlling rotational speed, treatment-fluid delivery, and suction.

In another method, the system performs exfoliation and treatment-fluid delivery while reducing the manual force required to move the treatment-face structures.

The operator activates the motorized treatment tip, treatment-fluid pump, and vacuum source.

The operator places distal contact portions of the treatment tip against skin with sufficient contact force to maintain contact while the electric motor supplies the treatment movement.

The treatment-fluid pump delivers treatment fluid while the rotating treatment face contacts the skin.

The controller changes rotational speed and treatment-fluid delivery according to stored commands rather than relying solely on changes in manual hand pressure.

In another method, the system increases contact between a treatment fluid and a skin region from which superficial stratum-corneum material has been removed.

The rotating treatment tip removes superficial material from a selected skin region.

Suction draws treatment fluid and removed material from the treated skin surface and superficial recesses into the vacuum-return path.

The treatment-fluid outlet supplies a treatment solution to the treated skin surface and to superficial pathways produced by abrasion.

The treatment tip can remain in contact with the selected region for a selected interval without requiring repetitive linear handpiece movement.

In another method, the electric motor reduces the manual movement required from an operator during a microdermabrasion treatment.

The electric motor rotates or oscillates the treatment tip during a localized treatment or a continuous treatment stroke.

Motorized movement reduces the need for manual scraping and repetitive cross-hatched strokes.

The vacuum-return path operates during motorized exfoliation to withdraw used treatment fluid and removed material.

The handpiece can be stabilized at a selected treatment location while the controller maintains a selected rotational speed, treatment-fluid-delivery rate, and suction level.

In another method, a single-use treatment tip reduces transfer of biological material between treatment procedures.

A new disposable treatment tip having a preapplied abrasive coating is installed before a treatment procedure.

The disposable treatment tip is removed after the treatment procedure and is not reused for another patient.

The integrated treatment-fluid path delivers treatment fluid without requiring manual application of treatment fluid after exfoliation.

The single-use treatment tip combines exfoliation, treatment-fluid delivery, and vacuum-assisted removal during one treatment procedure.

In another method, a controller changes treatment-tip speed according to a selected treatment program or measured motor load.

The controller operates the electric motor at one of multiple stored rotational-speed settings.

The operator or controller selects a rotational-speed setting for a treatment-tip structure and treatment region.

The motor-current sensor and rotational-speed sensor provide measured values used by the controller to reduce rotational speed when the filtered motor-current value exceeds a stored motor-current threshold and the measured rotational speed decreases by more than a stored rotational-speed amount.

The controller applies stored motor-speed, treatment-fluid-delivery, and suction commands without requiring the operator to change each command manually during the response interval.

### Additional Treatment Methods

In an additional method, the treatment tip 222 is placed against a treatment region, treatment fluid is supplied through the fluid outlet 244, suction moves the treatment fluid through a recessed passage across the skin-contact face and into the vacuum inlet 246, and the treatment tip 222 rotates while treatment fluid remains in the recessed passage. Rotation moves the treatment fluid and the treatment-face structures across the skin, and the vacuum-return stream removes the treatment fluid and material released from the skin.

The method may include measuring motor current more than 100 times per second, measuring rotational speed at a selected measurement rate, calculating a filtered motor-current value from four to sixty-four successive motor-current samples, and comparing the filtered motor-current value and measured rotational speed with stored numerical thresholds. When the filtered motor-current value rises by more than a stored amount and measured rotational speed falls by more than a stored amount for more than one successive comparison, the processor 212 sends a lower rotational-speed command to the motor driver 268 and a higher treatment-fluid-delivery command to the pump driver 274.

The method can be performed while translating the handpiece 216 in a continuous linear stroke or while maintaining the handpiece at a stationary treatment location. The selected treatment face can be grooved, pinned or nodular, planar, abrasively coated, or formed with a combination of those structures.

### Calibrated Motor-Current and Rotational-Speed Control

FIG. 17 depicts the processor 212, memory 272, motor driver 268, motor-current sensor 270, rotational-speed sensor 278, pump driver 274, variable-rate treatment-fluid pump 206, vacuum source 204, user control 276, and status indicator 282. The memory 272 stores numerical values and executable instructions used to acquire sensor samples, calculate comparison values, issue motor and pump commands, measure elapsed intervals, and issue a release command.

A baseline motor-current value is the arithmetic mean or weighted mean of a plurality of motor-current samples acquired while the treatment tip 222 rotates at a first commanded rotational speed, the pump 206 operates at a first commanded treatment-fluid delivery rate, the vacuum source 204 operates at a first commanded suction setting, and the treatment tip is spaced from the skin. A baseline rotational-speed value is calculated from rotational-speed samples acquired during the same baseline interval.

A calibration input from the user control 276 may start the baseline interval. The processor 212 may first operate the pump 206 and vacuum source 204 until treatment fluid passes from the fluid outlet 244, across the treatment face, through the vacuum inlet 246, and into the vacuum-return path. After the fluid path is primed, the processor acquires at least four successive motor-current samples and at least four successive rotational-speed samples and stores the resulting baseline values for the treatment session.

During treatment, the motor-current sensor 270 supplies successive motor-current samples and the rotational-speed sensor 278 supplies successive measured rotational-speed values. The processor 212 calculates a filtered motor-current value from a stored number of successive samples. The processor subtracts the baseline motor-current value from the filtered motor-current value to obtain a motor-current increase and subtracts the measured rotational speed from the commanded rotational speed to obtain a rotational-speed decrease.

The memory 272 stores a positive motor-current-increase threshold, a positive rotational-speed-decrease threshold, a required comparison count greater than one, a reset motor-current-increase threshold lower than the positive motor-current-increase threshold, a reset rotational-speed difference lower than the positive rotational-speed-decrease threshold, a nonzero response interval, a nonzero release interval, a positive minimum motor-current reduction, a motor-stop-current increment, a motor-stop threshold, a minimum rotational-speed command, a maximum pump-delivery command, a selected suction command, and a release-suction command that produces less suction than the selected suction command. The processor can set the motor-stop threshold equal to the baseline motor-current value plus the motor-stop-current increment. The thresholds, counts, intervals, and commands can be stored separately for different treatment-tip structures, treatment fluids, and treatment modes.

The memory 272 stores motor-current values in amperes or in digital values calibrated to amperes, rotational-speed values in revolutions per minute, treatment-fluid-delivery values as pump commands or milliliters per minute, suction values as actuator commands or pressure values, response and release intervals in milliseconds or seconds, and the required comparison count as an integer.

Before treatment begins, the processor 212 may verify that the baseline rotational-speed value differs from the first commanded rotational speed by no more than a stored baseline-speed tolerance and that the difference between the largest and smallest baseline motor-current samples does not exceed a stored baseline-current range. If either verification fails, the processor may stop the electric motor 218 or prevent the motor driver 268 from starting the electric motor and may activate the status indicator 282.

The processor 212 changes the motor and pump commands only after the motor-current increase exceeds the motor-current-increase threshold and the rotational-speed decrease exceeds the rotational-speed-decrease threshold for a stored number of successive comparisons. Requiring both measured conditions prevents an isolated current fluctuation or an isolated speed measurement from initiating the command change.

After both stored thresholds have been exceeded for the required comparison count, the processor 212 stores the filtered motor-current value from the last comparison as a triggering motor-current value. Before acquiring a subsequent motor-current sample, the processor sends a second rotational-speed command to the motor driver 268, sends a second pump-delivery command to the pump driver 274, and begins the response interval. The second rotational-speed command specifies a lower rotational speed than the first rotational-speed command, and the second pump-delivery command specifies a higher treatment-fluid delivery rate than the first pump-delivery command. The processor maintains the selected suction command during the response interval.

The processor 212 records the triggering motor-current value, the time at which the second rotational-speed command is issued, the time at which the second treatment-fluid-delivery command is issued, and the time at which the response interval ends. The post-response filtered motor-current value is calculated from samples acquired after issuance of both second commands.

At the end of the response interval, the processor 212 calculates a post-response filtered motor-current value and subtracts that value from the triggering motor-current value. When the resulting motor-current reduction equals or exceeds the stored positive minimum motor-current reduction, the processor retains the second rotational-speed command and the second pump-delivery command while the motor-current increase remains above the reset motor-current-increase threshold or the rotational-speed decrease remains above the reset rotational-speed difference.

When the motor-current reduction is smaller than the stored positive minimum motor-current reduction, or when the post-response filtered motor-current value equals or exceeds the motor-stop threshold, the processor 212 stops the electric motor 218, sends the release-suction command to the vacuum-control actuator 280, continues the second pump-delivery command for the nonzero release interval, and activates the status indicator 282. The lower suction and continued treatment-fluid delivery reduce the suction force holding the treatment tip against the skin and supply treatment fluid to the recessed passage while the treatment tip is released from the skin.

After the motor-current increase remains below the reset motor-current-increase threshold and the rotational-speed decrease remains below the reset rotational-speed difference for a stored number of successive comparisons, the processor 212 may send successive rotational-speed commands that increase in stored increments from the second rotational-speed command toward the first rotational-speed command and may send successive pump-delivery commands that decrease in stored increments from the second pump-delivery command toward the first pump-delivery command. The processor does not command a speed below the minimum rotational-speed command or a delivery rate above the maximum pump-delivery command.

The handpiece 216 can include the motor compartment 228 bounded by the fluid-tight wall 229. The first treatment-fluid conduit 232 has a noncircular transverse cross section bounded by a concave inner wall facing the motor compartment 228 and a convex outer wall facing the exterior wall of the handpiece housing 217. The bypass conduit 234 connects the first treatment-fluid conduit 232 with the core inlet 233 while extending beside the motor compartment 228. The vacuum-return passage 235 is physically separated from the first treatment-fluid conduit 232, the bypass conduit 234, and the motor compartment 228 by the fluid-tight wall 229.

FIG. 16A shows the proximal connector 224, distal opening 226, motor compartment 228, fluid-tight wall 229, electric motor 218, motor driver 268, treatment-fluid container 231, treatment-fluid-container interface 230, puncturing member 290, annular container seal 292, electrical connector 288, motor-current sensor 270, rotational-speed sensor 278, treatment-fluid supply seal 294, rotary seal 296, and vacuum-return passage 235 in the same longitudinal section. FIG. 16B shows the physical separation among the first treatment-fluid conduit 232, bypass conduit 234, vacuum-return passage 235, and motor compartment 228.

During rotation, the fluid outlet 244 supplies treatment fluid to a region of the skin-contact face 242 that is spaced from the vacuum inlet 246. Raised ridges 250, recessed channels 252, spaces between pins or nodules 254, an annular recess surrounding the planar treatment face 256, or molded channels in the abrasive treatment face 258 define a physical path from the outlet region to the inlet region. Rotation moves the treatment fluid tangentially, and the recessed portions redirect portions of the treatment fluid radially or circumferentially before the vacuum-return path removes the treatment fluid and material released from the skin.

FIG. 17 shows the treatment-fluid reservoir 208 and waste receptacle 210 at opposite ends of the fluid path, the pump driver 274 connected to the variable-rate treatment-fluid pump 206, the vacuum-control actuator 280 connected to the vacuum source 204, the signal-conditioning circuit 284 and analog-to-digital converter 286 connected between the motor-current sensor 270 and processor 212, and the status indicator 282 connected to the processor 212.

The figures are schematic and are not necessarily drawn to scale. Dimensions, materials, component locations, and operating values can be changed while retaining the described treatment-fluid paths, vacuum-return paths, rotating treatment surfaces, sensors, and controller operations. A structure described for one embodiment can be combined with a physically compatible structure described for another embodiment.

### Additional Motion-Source Embodiments

In some embodiments, the motion source includes a vibrational actuator mechanically coupled to the removable treatment tip or to the plurality of distal contact portions, the vibrational actuator configured to oscillate the plurality of distal contact portions along a path transverse to the skin-contact face. The vibrational actuator can include an electromagnetic coil-and-armature assembly, an eccentric-mass motor, or a piezoelectric element supported within the handpiece and mechanically linked to the removable treatment tip by a shaft, a diaphragm, or a coupling member. The vibrational actuator oscillates the distal contact portions at a frequency and amplitude selected to sequentially engage and disengage the distal contact portions with the skin at spaced-apart locations surrounding the treatment region, producing the sequential compressive engagement of tissue described in connection with FIG. 16C, without requiring continuous rotation of the treatment tip about a rotational axis.

In some embodiments, the motion source includes an acoustic transducer mechanically coupled to the removable treatment tip or to the plurality of distal contact portions, the acoustic transducer configured to impart mechanical vibration directly to the distal contact portions and thereby physically oscillate the distal contact portions against the skin. The acoustic transducer can include a piezoelectric element, a magnetostrictive element, or a voice-coil element supported within the handpiece and mechanically coupled to the removable treatment tip. This mechanical coupling is distinct from, and operates independently of, delivery of an electrical current to skin through an electrode; the acoustic transducer described in this paragraph produces physical movement of the distal contact portions rather than delivering an electrical signal to tissue. The acoustic transducer can operate at a frequency selected to produce the sequential compressive engagement of tissue and the tangential movement of treatment fluid described elsewhere in this disclosure.

In some embodiments, the motion source includes a reciprocating actuator mechanically coupled to the removable treatment tip and configured to reciprocate the treatment tip, or the plurality of distal contact portions, along a linear or arcuate path. The reciprocating actuator can include a solenoid, a cam-and-follower assembly, or a crank mechanism supported within the handpiece. The reciprocating actuator operates at a frequency and stroke length selected so that the reciprocating movement sequentially engages and disengages the distal contact portions with the skin at spaced-apart locations surrounding the treatment region, producing the sequential compressive engagement of tissue described in connection with FIG. 16C. The selected frequency and stroke length distinguish the reciprocating movement described in this paragraph from a reciprocating rotation of an abrading structure that does not produce that sequential compressive engagement.

In some embodiments, the motion source combines two or more of an electric motor coupled to rotate the treatment tip, a vibrational actuator, an acoustic transducer, and a reciprocating actuator, operable concurrently or sequentially to impart the repetitive relative movement described above. Each combination produces the sequential compressive engagement of tissue and the tangential movement of treatment fluid described elsewhere in this disclosure.

What is claimed is:

1. A skin-treatment system, comprising:
a treatment tip having a skin-contact face configured for contact with skin, the skin-contact face including a plurality of distal contact portions spaced apart from one another, a fluid outlet, a vacuum inlet spaced from the fluid outlet, and a recessed passage extending across the skin-contact face between the fluid outlet and the vacuum inlet;
a motion source coupled to the treatment tip and configured to impart repetitive relative movement between the plurality of distal contact portions and the skin during contact between the skin-contact face and the skin;
a fluid-delivery subsystem coupled to the fluid outlet and configured to deliver treatment fluid to the fluid outlet during the repetitive relative movement; and
a suction subsystem coupled to the vacuum inlet and configured to apply suction at the vacuum inlet during the repetitive relative movement;
wherein the repetitive relative movement sequentially engages and disengages individual ones of the plurality of distal contact portions with the skin at spaced-apart locations surrounding a treatment region, producing a sequential compressive engagement of tissue at the treatment region; and wherein the treatment fluid delivered at the fluid outlet and the suction applied at the vacuum inlet move material released from the treatment region by the sequential compressive engagement through the recessed passage toward the vacuum inlet.

2. The skin-treatment system of claim 1, wherein the motion source comprises one of the following: an electric motor coupled to rotate the treatment tip about a rotational axis; a vibrational actuator coupled to oscillate at least the plurality of distal contact portions along a path transverse to the skin-contact face; an acoustic transducer mechanically coupled to impart vibration directly to at least the plurality of distal contact portions, causing physical oscillation of the distal contact portions against the skin; or a reciprocating actuator configured to reciprocate the treatment tip along a linear or arcuate path at a frequency and stroke length selected to produce the sequential compressive engagement of tissue.

3. The skin-treatment system of claim 1, wherein the repetitive relative movement further moves the treatment fluid tangentially across the recessed passage relative to the treatment region, separates native surface lipid from the treatment region, and divides the separated surface lipid into droplets, films, or particles carried in the treatment fluid toward the vacuum inlet, and wherein, when the treatment region includes a follicular opening, the sequential compressive engagement presses and releases tissue on different, circumferentially spaced sides of the follicular opening, displacing material from the follicular opening toward the recessed passage.

4. The skin-treatment system of claim 1, wherein the motion source comprises an electric motor, a motor driver connected to the electric motor, and a rotatable drive member driven by the electric motor, the treatment tip being coupled to the rotatable drive member and the skin-contact face being rotatable about a rotational axis;
wherein the fluid-delivery subsystem comprises a treatment-fluid reservoir, a variable-rate treatment-fluid pump connected to the treatment-fluid reservoir, and a pump driver connected to the variable-rate treatment-fluid pump;
wherein the suction subsystem comprises a vacuum source and a waste receptacle connected by a vacuum-return path, and a vacuum-control actuator connected to the vacuum source and configured to change suction in response to a suction command;
the system further comprising a motor-current sensor connected in a supply circuit of the electric motor, a rotational-speed sensor, a user control that provides a calibration input, and an electronic controller including a processor and a memory, the memory storing executable instructions, a first rotational-speed command, a second rotational-speed command specifying a lower rotational speed than the first rotational-speed command, a first pump-delivery command, a second pump-delivery command specifying a higher treatment-fluid delivery rate than the first pump-delivery command, a positive motor-current-increase threshold, a positive rotational-speed-decrease threshold, a required comparison count greater than one, a nonzero response interval, a nonzero release interval, a positive minimum motor-current reduction, a motor-stop threshold, a selected suction command, and a release-suction command that produces less suction than the selected suction command,
wherein the executable instructions cause the processor to:
in response to the calibration input received while the treatment tip is spaced from skin, operate the electric motor according to the first rotational-speed command, operate the variable-rate treatment-fluid pump according to the first pump-delivery command, operate the vacuum source under control of the vacuum-control actuator according to the selected suction command, acquire a plurality of motor-current samples, and calculate a baseline motor-current value from the plurality of motor-current samples;
during contact between the skin-contact face and the skin, calculate a filtered motor-current value, subtract the baseline motor-current value from the filtered motor-current value to obtain a motor-current increase, receive a measured rotational speed from the rotational-speed sensor, and subtract the measured rotational speed from a rotational speed specified by the first rotational-speed command to obtain a rotational-speed decrease;
after the motor-current increase exceeds the positive motor-current-increase threshold and the rotational-speed decrease exceeds the positive rotational-speed-decrease threshold for the required comparison count of successive comparisons, store the filtered motor-current value from the last comparison as a triggering motor-current value and, before acquiring a subsequent motor-current sample, send the second rotational-speed command to the motor driver, send the second pump-delivery command to the pump driver, begin the nonzero response interval, and maintain the selected suction command during the nonzero response interval;
after expiration of the nonzero response interval, calculate a post-response filtered motor-current value and determine whether a difference between the triggering motor-current value and the post-response filtered motor-current value equals or exceeds the positive minimum motor-current reduction; and
when the difference is smaller than the positive minimum motor-current reduction, or when the post-response filtered motor-current value equals or exceeds the motor-stop threshold, stop the electric motor, operate the vacuum-control actuator according to the release-suction command, and continue operation of the variable-rate treatment-fluid pump according to the second pump-delivery command for the nonzero release interval.

5. The skin-treatment system of claim 4, wherein the memory further stores a reset motor-current-increase threshold lower than the positive motor-current-increase threshold and a reset rotational-speed difference lower than the positive rotational-speed-decrease threshold, and the executable instructions cause the processor, after the motor-current increase remains below the reset motor-current-increase threshold and the rotational-speed decrease remains below the reset rotational-speed difference for a stored number of successive comparisons, to send successive rotational-speed commands that increase in stored increments from the second rotational-speed command toward the first rotational-speed command and to send successive pump-delivery commands that decrease in stored increments from the second pump-delivery command toward the first pump-delivery command.

6. The skin-treatment system of claim 4, wherein the motor-current sensor includes either a current-sense resistor having a known resistance and a differential voltage-measurement circuit or a Hall-effect current sensor, an analog-to-digital converter supplies the motor-current samples to the processor at a rate greater than 100 samples per second, the processor calculates motor current from an output of the motor-current sensor, the processor calculates the filtered motor-current value from four to sixty-four successive motor-current samples, and the motor driver operates the electric motor at commanded rotational speeds from 10 revolutions per minute to 2,000 revolutions per minute.

7. The skin-treatment system of claim 4, further comprising a status indicator, wherein the memory stores a motor-stop-current increment greater than the positive motor-current-increase threshold, the processor sets the motor-stop threshold equal to the baseline motor-current value plus the motor-stop-current increment, and the executable instructions cause the processor to stop the electric motor, operate the vacuum-control actuator according to the release-suction command, continue treatment-fluid delivery for the nonzero release interval, and activate the status indicator when the post-response filtered motor-current value equals or exceeds the motor-stop threshold.

8. The skin-treatment system of claim 4, wherein the treatment tip includes an inner structure containing the fluid outlet and the vacuum inlet, an intermediate annular structure carrying the skin-contact face, and an outer annular structure carrying at least one electrode, the inner structure, intermediate annular structure, and outer annular structure being mechanically removable from the electric motor, and wherein the skin-contact face comprises one of the following: raised ridges separated by recessed channels, wherein an inclined sidewall of each raised ridge forms an angle from 35 degrees to 60 degrees relative to a plane containing distal ends of the raised ridges, each recessed channel has a depth measured from that plane to a floor of the recessed channel from 0.2 millimeter to 0.7 millimeter, adjacent raised ridges have a center-to-center spacing from 0.5 millimeter to 1.2 millimeters, a first recessed channel extends from an outlet region toward an inlet region along a first direction, and a second recessed channel extends from the outlet region toward the inlet region along a second direction different from the first direction; projecting pins or projecting nodules arranged in circumferentially offset rows, each projecting pin or projecting nodule having a base joined to the intermediate annular structure and a distal contact end spaced from the base, wherein spaces between adjacent projecting pins or projecting nodules form portions of the recessed passage between the outlet region and the inlet region; or emery particles bonded to a plastic substrate by an epoxy resin, wherein the epoxy resin and the emery particles are applied electrostatically and the epoxy resin is cured at 80 degrees Celsius for two hours.

9. The skin-treatment system of claim 4, wherein the system further comprises a motor compartment, a removable treatment-fluid container supported distal to the motor compartment, a container interface including a puncturing member and an annular seal that contacts a closure of the removable treatment-fluid container, a first conduit having a noncircular cross section bounded by a concave wall facing and partially surrounding a circumferential portion of the motor compartment and a convex wall facing an exterior wall of a handpiece housing, and a bypass conduit connecting the first conduit with a core inlet while extending beside the motor compartment, wherein, when a longitudinal handpiece axis is horizontal, a longitudinal container axis of the removable treatment-fluid container forms an acute angle with the longitudinal handpiece axis and descends toward the treatment tip, and wherein fluid-tight walls separate the first conduit and the bypass conduit from the motor compartment.

10. A method of treating skin, comprising:
positioning a treatment tip against a treatment region of skin, the treatment tip having a skin-contact face including a plurality of distal contact portions spaced apart from one another, a fluid outlet, a vacuum inlet spaced from the fluid outlet, and a recessed passage extending across the skin-contact face between the fluid outlet and the vacuum inlet;
imparting repetitive relative movement between the plurality of distal contact portions and the skin while the skin-contact face contacts the treatment region;
delivering treatment fluid to the fluid outlet during the repetitive relative movement;
applying suction at the vacuum inlet during the repetitive relative movement;
sequentially engaging and disengaging individual ones of the plurality of distal contact portions with the skin at spaced-apart locations surrounding the treatment region via the repetitive relative movement, thereby compressing and releasing tissue at the treatment region; and
withdrawing, through the vacuum inlet, material released from the treatment region by the compressing and releasing, together with the treatment fluid.

11. The method of claim 10, wherein imparting the repetitive relative movement comprises one of the following: rotating the treatment tip at a speed selected to shear native surface lipid from the treatment region without the plurality of distal contact portions penetrating a stratum corneum of the skin; vibrating at least a portion of the treatment tip along a path transverse to the skin-contact face; acoustically driving at least a portion of the treatment tip to mechanically oscillate against the skin; or reciprocating at least a portion of the treatment tip along a linear or arcuate path at a frequency and stroke length selected to produce the sequential compressive engagement of tissue.

12. The method of claim 10, wherein the treatment region includes a follicular opening, and the sequentially engaging and disengaging presses and releases tissue on different, circumferentially spaced sides of the follicular opening.

13. The method of claim 11, wherein imparting the repetitive relative movement comprises rotating the treatment tip, the method further comprising moving the treatment tip across the treatment region in a continuous linear stroke while rotating the treatment tip, delivering the treatment fluid, and applying suction, wherein the rotating moves the treatment fluid tangentially through the recessed passage relative to the treatment region, separates native surface lipid from the treatment region, divides the separated surface lipid into droplets, films, or particles carried in the treatment fluid, and moves the treatment fluid and the separated surface lipid through the vacuum inlet.

14. The method of claim 11, wherein imparting the repetitive relative movement comprises rotating the treatment tip, the method further comprising holding the treatment tip at one treatment location while rotating the treatment tip, delivering the treatment fluid, and applying suction, wherein circumferentially spaced distal contact portions sequentially press and release skin on different sides of a follicular opening and the treatment fluid and suction move material released from the follicular opening toward the vacuum inlet.

15. The method of claim 10, wherein imparting the repetitive relative movement comprises rotating the treatment tip via an electric motor, the method further comprising:
priming a treatment-fluid path by operating a treatment-fluid pump and a vacuum source to move treatment fluid from the fluid outlet, across the recessed passage, and into the vacuum inlet;
while the skin-contact face is spaced from skin, rotating the treatment tip according to a first rotational-speed command, operating the treatment-fluid pump according to a first pump-delivery command, operating the vacuum source under control of a vacuum-control actuator according to a selected suction command, acquiring a plurality of motor-current samples and a plurality of rotational-speed samples, calculating a baseline motor-current value from the plurality of motor-current samples, calculating a baseline rotational-speed value from the plurality of rotational-speed samples, and confirming that the baseline rotational-speed value is within a stored tolerance of the first rotational-speed command;
wherein the positioning of the treatment tip against the treatment region maintains treatment fluid in the recessed passage whose floor remains spaced from the treatment region, and the method further comprises continuing rotation of the treatment tip, delivery of the treatment fluid, and application of suction during that positioning;
during contact with the treatment region, calculating a filtered motor-current value, subtracting the baseline motor-current value from the filtered motor-current value to obtain a motor-current increase, measuring a rotational speed of the treatment tip, and subtracting the measured rotational speed from a rotational speed specified by the first rotational-speed command to obtain a rotational-speed decrease;
after the motor-current increase exceeds a stored positive motor-current-increase threshold and the rotational-speed decrease exceeds a stored positive rotational-speed-decrease threshold for a stored number of successive comparisons greater than one, storing the filtered motor-current value from the last comparison as a triggering motor-current value and, before acquiring a subsequent motor-current sample, sending a second rotational-speed command specifying a lower rotational speed, sending a second pump-delivery command to a pump driver, the second pump-delivery command specifying a higher treatment-fluid delivery rate, beginning a stored nonzero response interval, and maintaining the selected suction command during the stored nonzero response interval;
after expiration of the stored nonzero response interval, calculating a post-response filtered motor-current value and determining whether a difference between the triggering motor-current value and the post-response filtered motor-current value equals or exceeds a stored positive minimum motor-current reduction; and
when the difference is smaller than the stored positive minimum motor-current reduction, or when the post-response filtered motor-current value equals or exceeds a stored motor-stop threshold, stopping rotation of the treatment tip, operating the vacuum-control actuator to produce a suction level lower than a suction level produced by the selected suction command, and continuing treatment-fluid delivery according to the second pump-delivery command for a stored nonzero release interval.

16. The method of claim 15, further comprising, after the motor-current increase remains below a stored reset motor-current-increase threshold and the rotational-speed decrease remains below a stored reset rotational-speed difference for a stored number of successive comparisons, sending successive rotational-speed commands that increase in stored increments from the second rotational-speed command toward the first rotational-speed command and sending successive pump-delivery commands that decrease in stored increments from the second pump-delivery command toward the first pump-delivery command.

17. The method of claim 15, wherein acquiring the plurality of motor-current samples comprises acquiring more than 100 motor-current samples per second, calculating each filtered motor-current value from a moving window containing four to sixty-four successive motor-current samples, and updating the moving window after each newly acquired motor-current sample.

18. A motorized skin-treatment handpiece assembly, comprising:
an elongate handpiece housing having a proximal connector, a distal opening, and a motor compartment bounded by a fluid-tight wall;
an electric motor and a motor driver positioned in the motor compartment, the motor driver being connected to the electric motor, and the electric motor having a rotatable drive member extending toward the distal opening;
a treatment-fluid container interface supported by the handpiece housing;
a first treatment-fluid conduit extending from the treatment-fluid container interface and having a noncircular cross section bounded by a concave inner wall that faces and partially surrounds a circumferential portion of the motor compartment and a convex outer wall that faces an exterior wall of the handpiece housing;
a bypass conduit connected to the first treatment-fluid conduit and extending beside the motor compartment to a core inlet at the distal opening;
a vacuum-return passage extending from the distal opening to the proximal connector and physically separated from the first treatment-fluid conduit, the bypass conduit, and the motor compartment by fluid-tight walls;
a removable treatment tip coupled to the rotatable drive member, the removable treatment tip including a fluid outlet connected to the core inlet, a vacuum inlet connected to the vacuum-return passage, a skin-contact face positioned between the fluid outlet and the vacuum inlet, distal contact portions positioned to touch skin, and a recessed passage having a floor and sidewalls and extending across the skin-contact face from an outlet region to an inlet region, the floor being spaced proximally from the distal contact portions;
a motor-current sensor connected in a supply circuit of the electric motor;
a rotational-speed sensor positioned to measure a rotational speed of the rotatable drive member; and
an electrical connector having a first conductor that carries motor-current measurements from the motor-current sensor, a second conductor that carries rotational-speed measurements from the rotational-speed sensor, a third conductor that carries motor commands to the motor driver, a fourth conductor that supplies electrical power to the motor driver, the motor-current sensor, and the rotational-speed sensor, and a fifth conductor that provides an electrical return path.

19. The motorized skin-treatment handpiece assembly of claim 18, wherein the treatment-fluid container interface supports a removable treatment-fluid container distal to the motor compartment, and, when a longitudinal axis of the handpiece housing is horizontal, a longitudinal axis of the removable treatment-fluid container forms an acute angle with the longitudinal axis of the handpiece housing and descends toward the distal opening, the treatment-fluid container interface includes a puncturing member and an annular seal that contacts a closure of the removable treatment-fluid container, and the first treatment-fluid conduit extends from the treatment-fluid container interface along a side of the motor compartment.

20. The motorized skin-treatment handpiece assembly of claim 18, wherein the removable treatment tip includes an inner structure containing the fluid outlet and the vacuum inlet, an intermediate annular structure carrying the skin-contact face, and an outer annular structure carrying at least one electrode, and the intermediate annular structure is removable and has one of the following skin-contact faces: raised ridges separated by recessed channels, each recessed channel connecting the outlet region with the inlet region, wherein an inclined sidewall of each raised ridge forms an angle from 35 degrees to 60 degrees relative to a plane containing distal ends of the raised ridges, each recessed channel has a depth measured from that plane to a floor of the recessed channel from 0.2 millimeter to 0.7 millimeter, adjacent raised ridges have a center-to-center spacing from 0.5 millimeter to 1.2 millimeters, and at least two recessed channels extend along different angular directions between the outlet region and the inlet region; a substrate and projecting pins or projecting nodules arranged in circumferentially offset rows, each projecting pin or projecting nodule having a base joined to the substrate and a distal contact end spaced from the base, wherein spaces between adjacent projecting pins or projecting nodules form portions of the recessed passage between the outlet region and the inlet region; a continuous planar contact land adjacent to an annular recessed passage; or abrasive particles bonded to a substrate by an adhesive layer.
